(19) United States
(12) Patent Application Publication
(10) Pub. No.: US 2005/0058619 A1
(43) Pub. Date: Mar. 17, 2005

(54) Polyether-modified polymers as leather auxiliaries

(76) Inventors: Juergen Reiners, Leverkusen (DE); Martin Kleban, Leverkusen (DE); Nils Brinkmann, Leverkusen (DE)

Correspondence Address: LANXESS CORPORATION;PATENT DEPARTMENT/ BLDG 14, 100 BAYER ROAD, PITTSBURGH, PA, 15205-9741, US

(21) Appl. No.: 10/941,792

(22) Filed: Sep. 15, 2004

(30) Foreign Application Priority Data
Sep. 17, 2003 (DE) 10342926.3

(51) Int. Cl. A61K 31/765; C08L 77/00

(52) U.S. Cl. 424/78.37; 525/432

(57) ABSTRACT

(Co)polymers which contain
a) structural units of the general formula I 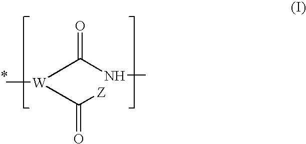 in which
W represents a trivalent radical from the group 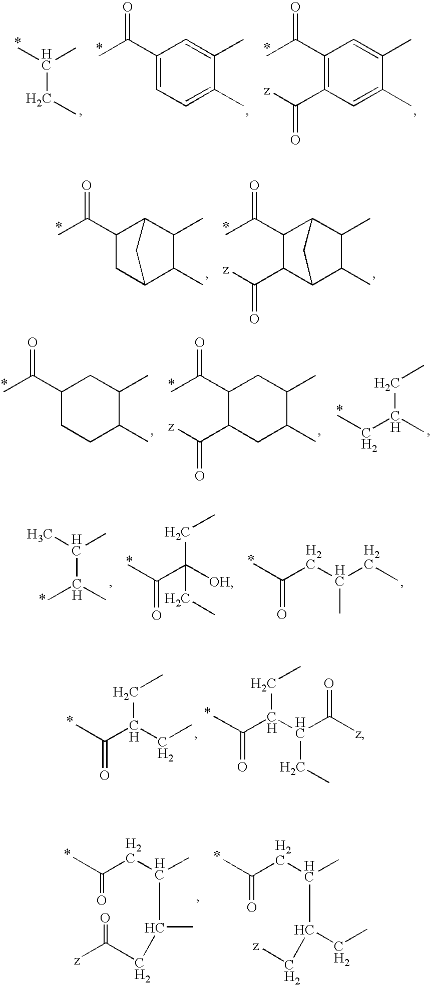 in which * indicates the orientation for the incorporation of the radical W into the formula I, and
Z represents the radicals —OH, —O⁻M⁺ or —N—R¹R², in which R¹ and R², independently of one another, represent hydrogen, optionally substituted alkyl radicals, alkenyl radicals, aralkyl radicals or cycloalkyl radicals, which may be interrupted by 0 atoms, N atoms, Si atoms or amido, carbonate, urethane, urea, allophanate, biuret or isocyanurate groups or mixtures thereof, and
M⁺ represents H⁺ or an alkali metal ion, an NH₄ ion or a primary, secondary, tertiary or quaternary aliphatic ammonium radical which preferably carries a C₁-C₂₂-alkyl or C₁-C₂₂-hydroxyalkyl group,
   b) at least 10 mol %, based on the units of the formula I, of structural units of the general formula Ia 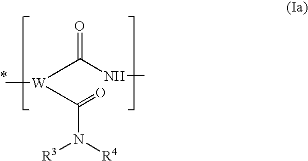 in which
R³ represents a hydrocarbon radical having C₁-C₆₀-atoms, preferably a saturated C₁-C₆₀-alkyl radical, in particular C₁₂-C₃₀-alkyl radical, and
R⁴ represents hydrogen or has the same meaning as R³, and
e) polyether units having an average molecular weight of 200-6000 g/mol.

The invention relates to polyether-modified polymers, a process for their preparation, dispersions containing them and the use thereof as leather auxiliaries.

By using these said leather auxiliaries, leathers having desirable properties are obtained. Such auxiliaries are used, as a rule, in the leather production process during tanning or retanning. For increasing the utility value of the leather, a balanced relationship of material properties is required in the case of many leather articles, for example in the case of upper leathers or furniture and automotive leathers. These include properties such as good fullness, a pleasantly soft hand, excellent grain smoothness and tight-grained character and an optimum tinctorial result.

DE-A-195 28 782 discloses polyaspartic acid derivatives by means of which the softness of the leather can be improved. With the use of these leather auxiliaries before, during and/or after the tanning or retanning, it is possible to establish certain leather properties. In this respect, it should be noted that the leathers should have sufficient mechanical strength and tight-grained character in spite of their softness. Moreover, the leather surface must not be tacky and should have a pleasant hand. These requirements are met when the auxiliaries are properly used. Nevertheless, these products are still in need of improvement with regard to the fullness effect, for example if firmer leather articles are desired.

EP 0 959 091 A1 and EP-A-959 090 disclose copolymers which are derived from hydrophobically modified polyaspartic acid ester amides and contain aspartic acid, aspartic ester, or aspartamide units, other proteinogenic or nonproteinogenic amino acid units and iminodisuccinate units as structural units. Such products are recommended as auxiliaries for various applications, the dispersing and complexing action in cosmetic emulsions and the foaming properties in surfactant formulations being of primary importance. Owing to their sensitivity to hydrolysis, the ester groups inevitably present in the product are not ideal for leather applications. The foaming action of the product described is a property which is likewise undesired for use in retanning liquors in leather production.

Other polymers containing aspartic acid units are known for other applications, for example from DE-A-19 545 678, EP-A-884 344, U.S. Pat. No. 5,292,858, DE-A-4 300 020, WO 94/01486, U.S. Pat. No. 5,286,810, U.S. Pat. No. 5,408,028, U.S. Pat. No. 5,357,004, U.S. Pat. No. 6,306,378 and WO 00/37023.

Accordingly, in spite of the large number of recommended compositions, there is still a need for products which impart not only plasticizing properties but also good fullness and good tinctorial properties to the leather and in particular permit high colour intensity and improve the levelness of the dyeing.

Surprisingly, it has now been found that certain polyether-modified polymers have the excellent effect described above on leather.

The invention relates to (co)polymers which contain
a) structural units of the general formula I 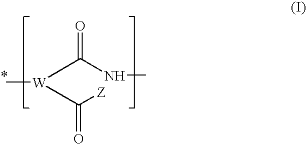
   in which
   W represents a trivalent radical from the group 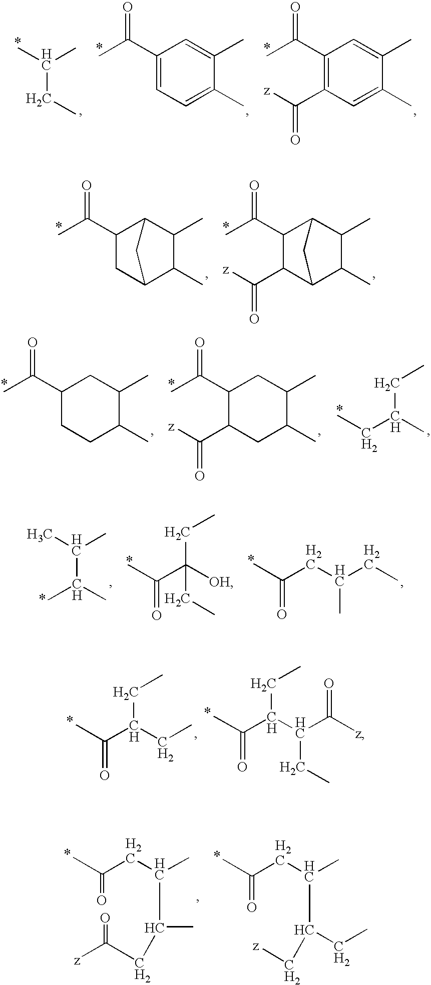
      in which
   * indicates the orientation for the incorporation of the radical W into the formula I, and
   Z represents the radicals —OH, —O⁻M⁺ or —N—R¹R², in which R¹ and R², independently of one another, represent hydrogen, optionally substituted alkyl radicals, alkenyl radicals, aralkyl radicals or cycloalkyl radicals, which may be interrupted by O atoms, N atoms, Si atoms or amido, carbonate, urethane, urea, allophanate, biuret or isocyanurate groups or mixtures thereof, and
   M⁺ represents H+ or an alkali metal ion, an NH₄ ion or a primary, secondary, tertiary or quaternary aliphatic ammonium radical which preferably carries a C₁-C₂₂-alkyl or C₁-C₂₂-hydroxyalkyl group,
b) at least 10 mol %, based on the units of the formula I, of structural units of the general formula Ia 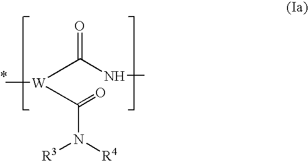
   in which
   R³ represents a hydrocarbon radical having C₁-C₆₀-atoms, preferably a saturated C₁-C₆₀-alkyl radical, in particular C₈-C₃₀-alkyl radical, and
   R⁴ represents hydrogen or has the same meaning as R³, and
c) polyether units having an average molecular weight of 200-6 000 g/mol.

The radicals W are derived, for example, from the hydrocarbon skeletons of the polycarboxylic acids from the following group or the anhydrides thereof:

Dicarboxylic acids or dicarboxylic anhydrides, such as maleic acid, maleic anhydride, fumaric acid, itaconic acid, itaconic anhydride, citraconic acid, citraconic anhydride, tricarboxylic acids or the anhydrides thereof, such as 1,2,3-propanetricarboxlic acid, citric acid, 1,2,4-cyclohexanetricarboxylic acid, 1,2,3-cyclohexanetricarboxylic acid, 2,3,5-norbornanetricarboxylic acid, trimellitic acid, trimellitic anhydride, and tetracarboxylic acids or the anhydrides or bisanhydrides thereof, such as 1,2,3,4-butanetetracarboxylic acid, 1,2,4,5-cyclohexanetetracarboxylic acid, 2,3,5,6-norbornanetetracaboxylic acid, 2,3,5,6-norbornanetetracarboxylic anhydride, pyromellitic acid, pyromellitic bisanhydride, etc. Maleic acid, fumaric acid, maleic anhydride or mixtures thereof with trimellitic anhydride or pyromellitic bisanhydride are preferred.

Suitable radicals M⁺ are, for example, hydrogen (H+), hydroxyethylammonium, bis(2-hydroxyethyl)-ammonium, tris(2-hydroxyethyl)ammonium, triethylammonium, tetraethylammonium, ammonium, butylammonium, N-methyl-N-bis(2-hydroxyethyl)-ammonium, N-dimethyl-N-2-hydroxyethyl)-ammonium, N-diethyl-N-(2-hydroxyethyl)-ammonium, benzyltrimethylammonium, morpholinium, hexadecylammonium, oleylammonium, octadecylammonium, and alkali metal ions, such as sodium, potassium, lithium. Sodium, potassium, hydrogen, ammonium, 2-hydroxyethylammonium, triethylammonium and tetraethylammonium are preferred.

Suitable nitrogen substituents R¹ and R² are, independently of one another, for example optionally substituted C₁-C₂₀₀-alkyl or C₂-C₂₀₀-alkenyl groups, such as methyl, ethyl, 2-hydroxyethyl, 2-hydroxypropyl, 3-hydroxypropyl, butyl, hexyl, octyl, 2-ethylhexyl, octenyl, decyl, undecyl, undecenyl, dodecyl, tetradecyl, hexadecyl, oleyl, octadecyl, 12-hydroxy-9-octadecenyl, eicosanyl, 3-heptamethyl-trisiloxanyl-propyl, N-2-(3-dimethyl-ethoxysilyl-1-propyl)amino-1-ethyl, N-2-(3-methyl-diethoxysilyl-1-propyl)amino-1-ethyl, N,N-dimethyl-2-aminoethyl, N,N-diethyl-2-aminoethyl, N,N-dimethyl-3-amino-1-propyl, N,N-dimethyl-2-amino-1-propyl, N,N-diethyl-3-amino-1-propyl, N,N-diethyl-2-amino-1-propyl, morpholinoethyl, morpholinopropyl, piperazinoethyl, ethoxy-ethyl, ethoxy-ethoxy-ethyl, butoxy-ethoxy-ethoxy-ethyl, 2-methoxy-ethyl, tetrahydrofurfuryl, 5-hydroxy-1-pentyl, benzyl, N,N-dimethyl-4-aminocyclohexyl, 2-sulphoethyl sodium or potassium salt, methoxycarbonyl-methyl or C₅-C₁₀-cycloalkyl radicals, such as cyclohexyl, C₁₂-C₂₀₀ radicals interrupted by oxygen atoms or ester, amido, urethane, urea, allophanate, biuret and carbonate groups, such as stearyloxyethyl, stearyloxyethoxyethyl, stearylcarbamoyloxyethyl, and radicals which are derived from polyethers, preferably based on ethylene oxide and/or propylene oxide, such as, for example, methoxy-(polyoxyethylene)-2-oxyprop-1-yl, methoxy-(polyoxyethylene-co-oxypropylene)-2-oxyprop-1-yl, ethoxy-(polyoxyethylene)-2-oxyeth-1-yl, ethoxy-(polyoxypropylene)-2-oxyprop-1-yl, ethoxy-(polyoxyethylene)-2-oxyprop-1-yl, and radicals of the formulae 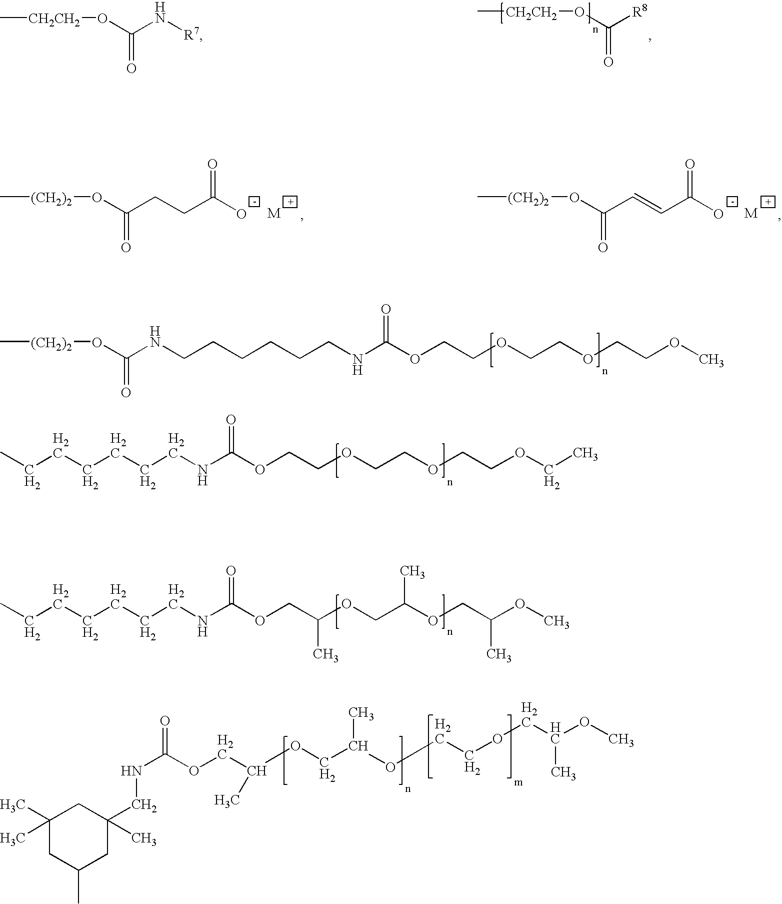
in which
R⁷ and R⁸ represent C₁-C₃₀-alkyl, C₂-C₃₀-alkenyl or C₅-C₁₀-cycloalkenyl,
n and m denote a number from 1 to 100, preferably from 1 to 50, and
M⁺ has the abovementioned meaning.

Further suitable radicals —NR¹R² are derived by abstraction of an H atom from monofunctional polyethers having primary amino groups based on oxirane, methyloxirane, tetrahyrofuran or mixtures thereof, it being possible for the polyether segments to be arranged randomly or in the form of blocks. Radicals which are derived from monofunctional aminopolyethers which have polyoxypropene and/or polyoxyethene units in any desired sequence are particularly preferred.

Such products are known per se and are obtainable, for example, under the name Jeffamine® M-600, Jeffamine® M-1000, Jeffamine® M-2070, Jeffamine® M-2005 (products of Huntsman).

Also suitable are radicals —NR¹R² which are derived by abstraction of an H atom from monofunctional polyethers having primary amino groups, which form in the reaction of polyisocyanates with monofunctional hydroxyl-terminated polyethers and subsequent hydrolysis. Instead of the hydrolysis, it is also possible to react the remaining isocyanate groups, for example, with excess primary diamines so that a free amino group remains.

The following may be mentioned as suitable polyisocyanates for the preparation of the monofunctional polyethers having primary amino groups: isophorone diisocyanate, bis(isocyanatocyclohexyl)-methane, xylylene diisocyanate, 1,4-bis(isocyanatomethyl)-cyclohexane, hexamethylene diisocyanate, toluene diisocyanate, diphenylmethane 4,4′-diisocyanate, diphenylmethane 2,4′-diisocyanate and those oligomers of the abovementioned diisocyanates which contain isocyanurate, biuret, allophanate, urea or uretdione groups.

The alkoxylation products which are known per se from polyurethane chemistry and are based on monofunctional initiator alcohols which are obtainable by polymerization of ethene oxide or propene oxide on, for example methanol, ethanol or butanol, etc. may be mentioned as monofunctional hydroxyl-functional polyethers.

The following may be mentioned as suitable diamines: 1,2-ethylenediamine, 1,2-propylenediamine, 1,3-propylenediamine, 1,4-butylenediamine, 1,2-dimethyl-1,2-ethylenediamine, 1,6-hexamethylenediamine, isophoronediamine, bis(aminocyclohexyl)methane and xylylenediamine.

Preferably, the structural unit of the formula I corresponds to the formula II 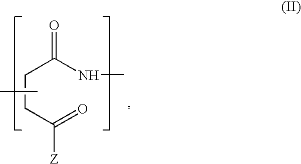
in which
Z has the abovementioned meaning.

Preferred structural units of the formula Ia correspond to the formula Ib 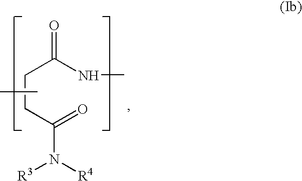
in which
R³ and R⁴ have the abovementioned meaning.

In a preferred embodiment, the polymer according to the invention contains units which contain ester groups and are of the formula 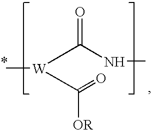
in which W has the above meaning and R represents any desired organic radical, in an amount of less than 5% by weight, in particular less than 2% by weight, preferably less than 1% by weight, based on copolymer.

Preferred units of the formula Ia, in particular units of the formula Ib, contain, as a radical of the formula —NR³R⁴, one which is derived from compounds of the formula HNR³R⁴, the following compounds being preferred as such: secondary, preferably primary, amines, such as 2-ethylhexylamine, 1-octylamine, 1-decylamine, 1-undecylamine, 1-dodecylamine, tetradecylamine, perfluorhexyl-ethylamine, N-aminoethyl-N-methyl-perfluoroctylsulphonamide, hexadecylamine, octadecylamine, octadecenylamine, tallow fatty amine, hydrogenated tallow fatty amine, oleylamine, 12-hydroxy-octadec-9-enylamine, eicosanylamine, dehydroabietylamine, stearoyloxypropylamine, 2-butyloctylamine, 4-octyl-hexadecylamine, docosanylamine (C₂₂), tetra-cosanylamine (C₂₄), triacontanylamine (C₃₀) and mixtures thereof.

—NR³R⁴ very particularly preferably represents C₈-to C₃₀-alkyl.

The polyether units can be incorporated in the main chain of the copolymer or in the side chain or in main chain and side chain.

Suitable polyether units (structural units from c)) are derived from monofunctional amino-polyethers for the side chain or from diamino-polyethers for the main chain.

The following may be mentioned by way of example as monofunctional amino-polyethers:

α-Methyl-ω-(3-aminopropyl)-polyoxyethene, α-methyl-ω-(2-aminopropyl)-polyoxyethene, α-methyl-ω-(3-aminopropyl)-polyoxypropene, α-methyl-ω-(2-aminopropyl)-polyoxypropene or the corresponding copolyethers which are derived from ethene oxide and propene oxide, and the ethoxylation and propoxylation products of C₁₂-C₁₄-fatty alcohols. Such polyetheramines are as a rule commercial raw materials and are obtainable, for example, under the name Jeffamine® M-600, Jeffamine® M-1000, Jeffamine® M-2005 or Jeffamine® M-2070 (M=monofunctional amine; number=average molecular weight) or Surfonamin ML-300 (products of Huntsman). Further suitable monofunctional amino-polyethers are obtainable, for example, by reacting a monofunctional OH-functional polyether with a diisocyanate in the molar ratio 1:1 or with an excess of diisocyanate, optionally freeing the resulting reaction product from excess diisocyanate and then hydrolyzing the isocyanate groups to the amine. Reaction products of propene oxide-polyethers or polyethylene oxide-polyethers or copolymers based on ethene oxide and propene oxide, which were initiated on monofunctional alcohols, such as methanol, ethanol, methoxyethanol, methoxy-diethylene glycol, butanol, dibutylglycol, etc., with diisocyanates, such as hexamethylene diisocyanate, isophorone diisocyanate, bis(isocyanatocyclohexyl)-methane, xylylene diisocyanate, toluene diisocyanate, etc. (preferably in the molar ratio NCO:OH of 2:1) and hydrolysis of the resulting NCO prepolymer to the monoamine or by reaction of the resulting NCO prepolymer with diamines to give a monoamine may be mentioned by way of example. Monofunctional polyetheramines which have a number average molar mass of 200 to 6 000 g/mol are preferred.

The following may be mentioned by way of example as diamino-polyethers:

α,ω-bis(3-aminopropyl)-polyoxypropene, α,ω-bis(2-amino-1-propyl)-polyoxypropene, α,ω-bis(2-amino-1-propyl)-poly(oxyethene-co-oxypropene), α,ω-bis(2-amino-1-ethyl)-polyoxyethene, α,ω-bis(2-amino-1-ethyl)-poly(oxyethene-co-oxypropene), α,ω-bis(2-amino-1-ethyl)-polyoxypropene, α,ω-bis(2-amino-1-propyl)-poly(oxyethene-co-oxybutene), α,ω-bis(2-amino-1-ethyl)-poly-(oxyethylene-co-oxybutene), α,ω-bis(4-amino-1-butyl)-polyoxybutene, α,ω-bis(4-amino-1-butyl)-poly(oxybutene-co-oxypropene), α,ω-bis(4-amino-1-butyl)-poly(oxybutene-co-oxyethene), α,ω-bis(6-amino-1-hexyl-carbamoyl)-polyoxybutene, α,ω-bis(6-amino-1-hexyl-carbamoyl)-polyoxyethene, α,ω-bis(6-amino-1-hexyl-carbamoyl)-polyoxypropene or mixtures thereof. Diaminopolyethers based on propene oxide and/or ethene oxide, having an average molar mass of 200-4 000 g/mol, are preferred. Such diamino-polyethers are generally commercially available and are obtainable, for example, under the name Jeffamine® D-230, Jeffamine® D-400, Jeffamine® D-2000 and Jeffamine® ED-600, Jeffamine® ED-2003, Jeffamine® XTJ 511 (products of Huntsman; D series=PO-based difunctional diaminopolyethers; ED series=EO/PO-based difunctional diamino-polyethers, number=average molecular weight). Furthermore preferred are diaminopolyethers which are obtainable by reaction of diisocyanates, such as hexamethylene diisocyanate, isophorone diisocyanate or bis-isocyanatocyclohexylmethane with hydroxyl-functional polyethers based on ethene oxide, propene oxide or tetrahydrofuran or the copolyethers or mixtures thereof in an NCO: OH molar ratio greater than 1:1 and subsequent hydrolysis of the terminal isocyanate group. Instead of the hydrolysis, a reaction of the terminal isocyanate group with diamines is also possible for obtaining suitable diamino-polyethers. In this case, the diamine must be used in excess.

In addition to the hydroxyl-functional polyethers mentioned in the preceding paragraph, other hydroxyl-terminated building blocks customary in polyurethane chemistry, such as polycarbonatediols, polyesterdiols, polyestercarbonatediols and polyethercarbonatediols, which are derived, for example, by reaction of the abovementioned hydroxyl-functional polyethers and other customary building blocks, such as ethylene glycol, propylene glycol, butanediol, neopentylglycol, hexanediol or caprolactone, by polycondensation with diphenyl carbonate or dimethyl carbonate (with elimination of phenol or methanol) and/or by polycondensation with adipic acid, glutaric acid, terephthalic acid or isophthalic acid (with elimination of water) or mixtures thereof, are also suitable as raw materials for the preparation of the diamino-polyethers, preferably those which contain polyether units incorporated in the polymer chain. Suitable diisocyanates and diamines for the reaction of these building blocks are the abovementioned diisocyanates and diamines. Particularly preferred reactants for this synthesis are polyethers based on oxirane and methyloxirane having two terminal hydroxyl groups, diisocyanates, such as hexamethylene diisocyanate and isophorone diisocyanate, and diamines, such as ethylenediamine, hexamethylenediamine and isophoronediamine.

A further possibility for the preparation of suitable diamino-functional polyethers is the base-catalyzed addition reaction of acrylonitrile with hydroxyl-terminated polyethers and subsequent hydrogenation of the nitrile groups to amino groups.

A further possibility is also the catalytic amination of the polyethers with ammonia.

Of course, all linear amino-terminated polyurethanes, polyureas or polyurethaneureas in the synthesis of which polyethers are incorporated in the main chain are suitable as raw materials for the preparation of the polyether-modified copolymers according to the invention. In addition to the polyethers based on ethene oxide and/or propene oxide and/or tetrahydrofuran or copolyethers or any desired mixtures thereof, these polyurethanes, polyureas or polyurethaneureas may also contain all other polymer diols (C₄-polyethers, polyesters, polycarbonates, polyestercarbonates), low molecular weight diols (butanediol, hexanediol, the adducts of ethylene oxide or propylene oxide with tallow fatty amine or hydrogenated tallow fatty amine), hydrophylizing agents (dimethylolpropionic acid, the adduct of sodium bisulphite with a 1,4-butenediol-initiated propylene oxide polyether having a molar mass between 300 and 1 000 g/mol), diamines (hydrazine hydrate, ethylenediamine, hexamethylenediamine, isophoronediamine, bis(aminocyclohexyl)methane, N2-aminoethyl)-2-aminoethane-sulphonic acid sodium salt, N-(2-aminoethyl)-2-aminopropionic acid sodium salt), bis-aminopropyl-terminated polysiloxanes having molar masses between 200 and 3 000 g/mol, etc., which are customary in polyurethane chemistry, as raw materials.

Diamines containing polyethene oxide and/or polypropene oxide units and having an average molecular weight of 200 to 6 000 g/mol.

The prefix “poly” in the abovementioned raw material designation preferably has a value such that the corresponding monoamines or diamines have a preferred molecular weight, determined as the number average, of 200 to 4 000 g/mol, preferably of 400 to 2 500 g/mol.

In a preferred embodiment, the proportion of units of the formula (I) where 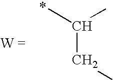
is more than 50 mol %, based on the sum of all units of the formula (I), in particular more than 80 mol %, very particularly preferably more than 90 mol %.

Preferred copolymers according to the invention contain structural units of the formulae (IIa) and (IIb)

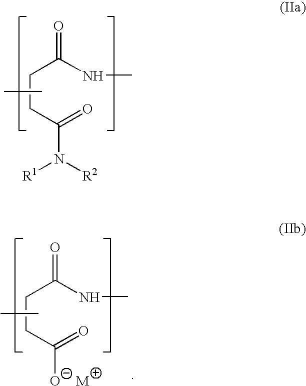

In addition, the copolymers according to the invention may contain, in the main chain, further divalent bridge members V differing from the units a), b) and c).

Preferred copolymers contain 0 to 10 mol % of such divalent bridge members V in the main chain, based on the sum of all units of the formula I, V representing a divalent radical which is derived from polycarboxylic acids, polyamines, lactams or aminocarboxylic acids. The bridge members V are preferably derived from polyamines, in particular diamines, or polycarboxylic acids, in particular dicarboxylic acids, and from aminocarboxylic acids or the lactams thereof.

These units may be contained in the main chain and then preferably serve for linking the units of the formula I, Ia, II, IIa, IIb to one another in any desired sequence. They may also serve for linking different polyether units which are incorporated in the main chain. It is also possible for a plurality of divalent radicals from the abovementioned group of the polycarboxylic acids, polyamines, lactams and aminocarboxylic acids to be condensed and thus incorporated in the form of longer bridge members.

Preferred bridge members V are derived from the following polyamines (V-1): ethylenediamine, 1,2- and 1,3-propylene diamine, 1,6-hexamethylenediamine, isophoronediamine, bis-aminocyclohexylmethane, diethylenetriamine, triethylenetetramine, bishexamethylenetriamine, diaminocyclohexane, xylylenediamine, bis-3-aminopropyl-ether, bis-aminomethyl-tricyclo[5.2.1.0^{2,6}]decane. Diamines, in particular ethylenediamine and propylenediamine, isophoronediamine and hexamethylenediamine are preferred.

Preferred bridge members V are derived from the following polycarboxylic acids (V-2): oxalic acid, malonic acid, succinic acid, glutaric acid, adipic acid, sebacic acid, tartaric acid, cyclohexane-1,2-dicarboxylic acid, cyclohexane-1,3-dicarboxylic acid, cyclohexane-1,4-dicarboxylic acid, 1-cyclohexene-1,2-dicarboxylic anhydride, 4-cyclohexene-1,2-dicarboxylic acid, 4-cyclohexene-1,2-dicarboxylic anhydride, 3-cyclohexene-1,2-dicarboxylic anhydride, norbornane-2,3-dicarboxylic acid, norbornane-2,3-dicarboxylic anhydride, 5-norbornene-2,3-dicarboxylic acid, phthalic acid, isophthalic acid, terephthalic acid, naphthalene-1,2-dicarboxylic acid. Dicarboxylic acids, in particular succinic acid, glutaric acid and adipic acid, are preferred.

Preferred bridge members V are derived from the following aminocarboxylic acids (V-3): glycine, alanine, aspartic acid, glutamic acid, other naturally occurring amino acids, aminopropionic acid, aminobutyric acid, aminohexanecarboxylic acid. Glycine, aspartic acid and glutamic acid are preferred.

Preferred bridge members V are derived from the following lactams (V-4): butyrolactam, caprolactam, laurolactam. Caprolactam is preferred.

Suitable longer bridge members V may also be obtained by condensation or cocondensation reactions from the abovementioned building blocks V-1 to V-4. In the preparation of the (co)polymers according to the invention, these bridge members are preferably incorporated into the main chain thereof, owing to the terminal amino groups and/or carboxyl groups.

The (co)polymers according to the invention preferably have, as terminal groups, the following radicals which can be defined by means of the starting material and are preferably derived from the optionally used polycarboxylic acids, dicarboxylic acids, polyamines, diamines, lactams, ammonia, amines, polyethers and the monomer building blocks used for the preparation of the monofunctional and difunctional polyethers. In particular, the terminal groups may be based on the following radicals: alkyl radicals, alkoxy radicals, alkylamino radicals, cycloalkylamino radicals or aliphatic amino-substituted aralkyl radicals, polyetheramino radicals, N-substituted maleimide radicals, OH, COOH, CONH₂ and NH₂ groups, and radicals which are derived from aspartic acid and derivatives thereof. Preferred terminal groups are, for example, hydroxyl, amino, carboxyl and salts thereof, carboxamide, N-substituted maleimide, N-substituted aspartamide, methyl, ethyl, butyl, methoxy, ethoxy, butoxy, butylamino, hexylamino, decylamino, dodecylamino, tetradecylamino, hexadecylamino, octadecylamino, eicosanylamino, N-methyl-N-octadecylamino, bis(octadecyl)amino, benzylamino and 2-hydroxy-ethylamino.

The polyether-modified copolymers according to the invention and having a molecular weight, determined as the number average, of 500 to 50 000, preferably 1 500 to 30 000, g/mol are particularly preferred, especially those which contain
5 to 35 mol % of structural units of the formula Ha and
15 to 90 mol % of structural units of the formula IIb,
based in each case on the sum of the structural units of the formula I.

Those copolymers according to the invention which contain 10 to 90% by weight, in particular 30 to 80% by weight, of structural units of the formula I, based in each case on the copolymer, are preferred.

The copolymer according to the invention preferably comprises more than 95% by weight, in particular more than 98% by weight, of the structural units of the formula I, Ia, the polyether units and optionally divalent bridge members.

In a particular embodiment—referred to below as A1 —the copolymer according to the invention comprises more than 95% by weight, in particular more than 98% by weight, of structural units of the formula I and polyether units,
i) 50 to 90 mol %, in particular 70 to 90 mol %, based on the sum of these two units, being accounted for by structural units of the formula I, of which at least 10 mol %, in particular 10 to 35 mol %, based on the units of the formula I, correspond to those of the formula Ia, in particular of the formula Ib, and
ii) 10 to 50 mol %, preferably 10 to 30 mol %, based on the sum of these two units, being accounted for by polyether units having an average molecular weight of 200 to 6 000 g/mol.

In a very particularly preferred embodiment A1, the radical —NR³R⁴ in formula Ia, in particular Ib, represents radicals which are derived from the following amines HNR³R⁴: dodecylamine, tetradecylamine, hexadecylamine, octadecylamine, hexadecenylamine, octadecenylamine, tallow fatty amine, hydrogenated tallow fatty amine, N-methyl-octadecylamine, branched aliphatic amines which are derived from the corresponding Guerbet alcohols, such as, for example, 2-butyl-1-octylamine, 2-hexyl-1-octylamine, 2-butyl-1-decylamine, 2-hexyl-1-decylamine, 2-octyl-1-decylamine, 2-hexyl-dodecylamine, 2-octyl-dodecylamine, 2-decyl-1-tetradecylamine, dodecyl-hexadecylamine, tetradecyl-octadecylamine, hexadecyl-octadecylamine, hexadecyl-eicosanylamine, and amino compounds which are obtained by reaction of Guerbet alcohols, such as, for example, 2-butyl-1-octanol, 2-hexyl-1-octanol, 2-butyl-1-decanol, 2-hexyl-1-decanol, 2-octyl-1-decanol, 2-hexyl-dodecanol, 2-octyl-dodecanol, 2-decyl-1-tetradecanol, dodecyl-hexadecanol, tetradecyl-octadecanol, hexadecyl-octadecanol or hexadecyl-eicosanol, with hexamethylene diisocyanate, isophorone diisocyanate, xylylene diisocyanate, cyclohexylene diisocyanate or bis-(isocyanatocyclohexyl)methane in the molar ratio 1:1 to 1.5:1 and subsequent hydrolysis of the NCO groups of by reaction of the NCO groups with excess diamine, or mixtures of the abovementioned amines HNR³R⁴.

In a very particularly preferred embodiment A1, the copolymer contains polyether units of the component ii) which are derived from diamines of the formula

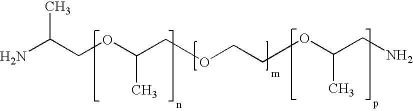

| | | | |
|---|---|---|---|
| Jeffamine | molar mass about 220 g/mol | m = 2.0 | n + p = 2.0 |
| XTJ 511: | | | |
| Jeffamine | molar mass about 600 g/mol | m = 9.0 | n + p = 3.6 |
| ED-600: | | | |
| Jeffamine | molar mass about 2000 g/mol | m = 38.7 | n + p = 6.0 |
| ED-2003: | | | |

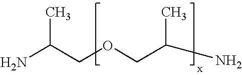

| | | |
|---|---|---|
| Jeffamine D-230: | molar mass about 230 g/mol | x = 2-3 |
| Jeffamine D-400: | molar mass about 400 g/mol | x = 5-6 |
| Jeffamine D-2000: | molar mass about 2000 g/mol | x = about 33 |
| Jeffamine D-4000: | molar mass about 4000 g/mol | x = about 68 |

In a likewise particular embodiment—referred to below as A2—the copolymer according to the invention comprises more than 95% by weight, in particular more than 98% by weight, of structural units of the formula I, of which
i) at least 10 mol %, in particular 10 to 50 mol %, based on the structural units of the formula I, correspond to the formula Ia, in particular Ib, and
ii) at least 10 mol %, in particular 10 to 50 mol %, based on the units of the formula I, correspond to the formula Ic, in particular the formula Id, 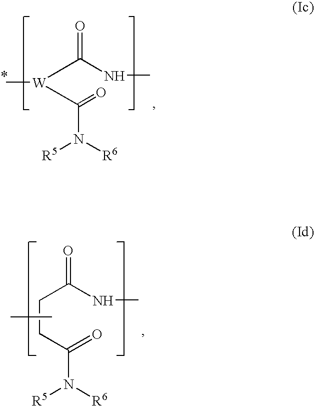
   in which
   W has the abovementioned meaning,
   R⁵ represents a radical which contains more than two ether groups derived from C₂-C₆-alkylene oxide units and which is optionally interrupted by urethane, carbonate, urea, biuret, allophanate, isocyanate, alkylene, cycloalkylene or aralkylene groups, and
   R⁶ represents hydrogen or has the meaning of R⁵.

In a preferred embodiment A2, the radical —NR³R⁴ in formula Ia, in particular Ib, has the same meaning as that stated above for the embodiment A1.

In a preferred embodiment A2, the radical —NR⁵R⁶ is derived from amines of the formula HNR⁵R⁶, which correspond to the abovementioned monofunctional amino-polyethers, in particular from primary amino-polyethers.

Particularly preferred are polyether units of the component ii), which are derived from the following monoamines: 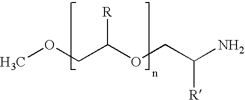
in which
R represents H or C₁-C₄-alkyl, in particular H or CH₃,
R′ denotes H or CH₃ and

n denotes a number from 5 to 50, in particular

| | | |
|---|---|---|
| Jeffamine XTJ-505 | molar mass about 600 g/mol | molar ratio PO |
| (M-600): | | |
| Jeffamine XTJ-506 | molar mass about 1000 g/mol | molar ratio PO |
| (M-1000): | | |
| Jeffamine XTJ-507 | molar mass about 2000 g/mol | molar ratio PO |
| (M-2005): | | |
| Jeffamine M-2070: | molar mass about 2000 g/mol | molar ratio PO |

and monofunctional polyether units which form by reaction of hexamethylene diisocyanate, isophorone diisocyanate, bis(4-isocyanatohexyl)-methane, toluene diisocyanate, diphenylmethane 4,4′-diisocyanate, diphenylmethane 2,4′-diisocyanate, xylylene diisocyanate or those oligomers of the abovementioned diisocyanates which contain isocyanurate, uretdione, allophanate or urea groups with monofunctional hydroxyl-terminated polyethers, the latter being obtained by alkoxylation of monofunctional initiator alcohols, such as methanol, ethanol or butanol, with ethene oxide, propene oxide or mixtures thereof, and subsequent hydrolysis of the remaining isocyanate group or reaction of the remaining isocyanate group with an excess of diamines, such as ethylenediamine, 1,2-propylenediamine, 1,3-propylenediamine, 1,4-butylenediamine, 1,6-hexamethylenediamine, isophoronediamine or bis(aminocyclohexyl)methane to give the monofunctional polyetheramine, such as, for example, 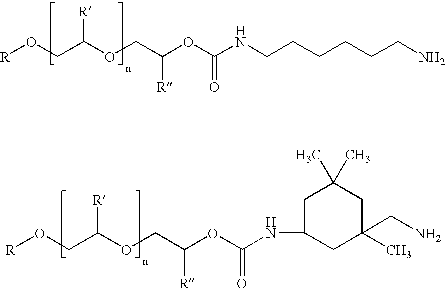
in which
R, R′ and n have the abovementioned meaning
   and
R′ represents H or CH₃.

The invention relates to a process for the preparation of the (co)polymers according to the invention, which is characterized in that
A-1) polycarboxylic acids or the anhydrides thereof selected from the group consisting of
   maleic acid, maleic anhydride, fumaric acid, itaconic acid, itaconic anhydride, citraconic acid, citraconic anhydride, 1,2,3-propanetricarboxylic acid, citric acid, 1,2,4-cyclohexanetricarboxylic acid, 1,2,3-cyclohexanetricarboxylic acid, 2,3,5-norbornanetricarboxylic acid, trimellitic acid, trimellitic anhydride, 1,2,3,4-butanetetracarboxylic acid, 1,2,4,5-cyclohexanetetracarboxylic acid, 2,3,5,6-norbornanetetracarboxylic acid, 2,3,5,6-norbornanetetracarboxylic anhydride, pyromellitic acid and pyromellitic bisanhydride
A-2) and optionally further polycarboxylic acids and/or anhydrides (V-2) differing from A-1) (for introduction of the units V)
A-3) and optionally aminocarboxylic acids (V-3) or lactams (V-4) (for introducing the bridge members V)
   are reacted with
B-1) ammonia
B-2) and optionally with diamines which contain polyether groups and have an average molar mass of 200-6000 g/mol
B-3) and optionally with additional polyamines (V-1) differing from B-2 (for introducing the bridge members V)
   to give polycarboxamide-ammonium salts and these
C) are condensed with elimination of water,
D-1) the polycondensate obtained is reacted with amines of the formula HNR³R⁴,
   in which R³ and R⁴ have the abovementioned meaning,
D-2) and optionally with amines of the formula HNR⁵R⁶ which contain polyether groups and have a molar mass of 200-6 000 g/mol
   in which R⁵ and R⁶ have the abovementioned meaning, and
E) the reaction product is then reacted with a ring-opening base in the presence of water,
   with the proviso that at least one of the two components B-2) or D-2) is used in the reaction.

Examples of suitable polycarboxylic acids of the component A-2 are: oxalic acid, malonic acid, succinic acid, glutaric acid, adipic acid, sebacic acid, tartaric acid, cyclohexane-1,2-dicarboxylic acid, cyclohexane-1,3-dicarboxylic acid, cyclohexane-1,4-dicarboxylic acid, 1-cyclohexene-1,2-dicarboxylic anhydride, 4-cyclohexene-1,2-dicarboxylic acid, 4-cyclohexene-1,2-dicarboxylic anhydride, 3-cyclohexene-1,2-dicarboxylic anhydride, norbornane-2,3-dicarboxylic acid, norbornane-2,3-dicarboxylic anhydride, 5-norbornene-2,3-dicarboxylic acid, phthalic acid, phthalic anhydride, isophthalic acid, terephthalic acid, naphthalene-1,2-dicarboxylic acid.

Examples of suitable polyamines of component B-2) are: ethylenediamine, 1,2-propylenediamine, 1,3-propylenediamine, 1,4-butylenediamine, 1,6-hexamethylenediamine, isophoronediamine, bis(aminocyclohexyl)methane, diethylenetriamine, triethylenetetramine, bishexamethylenetriamine, the various isomers of diaminocyclohexane, xylylenediamine, bis-3-aminopropyl ether, bis-aminomethyl-tricyclo[5.2.1.0 ^{2,6}]decane.

Examples of suitable aminocarboxylic acids of the component A-3) are: alanine, glycine, glutamic acid, aspartic acid or other naturally occurring amino acids, aminopropionic acid, aminobutyric acid, aminohexanoic acid.

Examples of suitable lactams of the component A-3) are:
butyrolactam, caprolactam, laurolactam.

The reaction of the components A-1) (optionally in the presence of A-2) and/or A-3)) with ammonia B-1), preferably aqueous ammonia solution, and optionally the polyetherdiamines B-2) and optionally polyamines B-3) to give the polycarboxamide-ammonium salts is effected in the temperature range between 5° C. and 100° C., preferably 20 to 80° C. The metering of the component B-2) is effected before, during or after the reaction with ammonia, preferably in the presence of solvents. Suitable solvents are N-methylpyrrolidone, pyrrolidone, ethylene glycol, dimethylacetamide, dimethylformamide, diethylene glycol, triethylene glycol, ethanol, butanol, n-propanol, isopropanol, ethoxy-propanol and butoxy-propanol. Particularly preferred solvents are ethylene glycol and diethylene glycol, pyrrolidone and N-methylpyrrolidone. Diethylene glycol is particularly preferred.

The condensation C) is preferably effected with heating of the reaction mixture obtained after the reaction of A-1) (optionally in the presence of A-2) and/or A-3)) with B-1) (and optionally B-2) and optionally B-3)) to temperatures between 70 and 200° C., preferably 90 to 170° C., the water from the ammonia solution added in the preceding step and the water of reaction formed in the polycondensation being distilled off. The polycondensation is preferably carried out under reduced pressure in the range between 0.1 mbar and 800 mbar.

The reaction of the resulting polycondensate with amines of the formula HNR³R⁴ D-1) and optionally with amines of the formula HNR⁵R⁶ D-2) which contain polyether groups is effected at temperatures above 120° C., preferably 120 to 170° C. The reaction is preferably effected in the presence of a solvent. The solvents already mentioned above are suitable for this purpose. Ethylene glycol, diethylene glycol, N-methylpyrrolidone and pyrrolidinone are preferred. Diethylene glycol is particularly preferred.

The reaction E) with a ring-opening base is effected during or after the dispersing in water. The dissolved melt of the reaction mixture which is obtained after the reaction with D-1) and optionally D-2) is preferably dispersed in water at 70 to 140° C. and at the same time the reaction with the ring-opening base is carried out, a dispersion of the copolymer being obtained. The amount of the base is preferably chosen so that a homogeneous, readily stirrable dispersion is obtained. Ring-opening base is preferably added in an amount such that the imide structural units still present in the copolymer after the reaction with D-1) and optionally D-2) are hydrolyzed.

The reaction E) is preferably effected in the presence of derivatives of C₁-C₁₈-moncarboxylic acids.

A process for the preparation of the (co)polymers according to the invention in which maleic acid or maleic anhydride is reacted with ammonia, optionally in the presence of diamines containing polyether groups, and is then condensed and the resulting polymer is reacted with an amine of the abovementioned formula HNR³R⁴ and optionally with an amine HNR⁵R⁶ containing polyether groups and the reaction product therefrom is reacted with a ring-opening base in the presence of water and optionally of a C₁-C₁₈-monocarboxylic acid is preferred.

More than 95% by weight, in particular more than 98% by weight of building block which are selected from the group consisting of maleic anhydride, maleic acid, fumaric acid, ammonia, diamines containing polyether groups and having an average molecular weight of 200 to 6 000 g/mol, amines of the formula HNR³R⁴ and amines of the formula HNR⁵R⁶ which contain polyether groups are particularly preferably used for the preparation of the products according to the invention.

The concomitant use of polyether-containing diamines in step B-2) preferably results in a polymer structure in which individual or a plurality of units of the formula I are linked to one another via polyether units, or results in a block-like structure, it being possible for the linkage to be effected at both ends via a carboxamide bond (head-to-head linkage) or at both ends in the manner of a Michael addition at double bonds still present in maleic acid units (tail-to-tail linkage) or via a mixture of the two bonding types (head-to-tail linkage). The groups I may change the orientation in the main chain of the copolymers. By using additional components (in A-2) and/or A-3) and/or B-3)), for example, divalent bridge members V form within the polymer chain.

Preferred polyether-modified polymers according to embodiment A1 have molecular weights, determined as the weight average by gel permeation chromatography (calibrated with polystyrene), of 500 to 30 000, preferably 1 000 to 20 000, in particular 1 000 to 10 000, g/mol. The structural units are preferably distributed in an alternating, block-like or random manner.

The polymers may also contain branching points which, for example, in the case of maleic anhydride, can form as a result of incorporation of iminodisuccinate radicals or as a result of incorporation of nitrilotrisuccinate radicals or with the concomitant use of tri- or tetracarboxylic acid or as a result of a Michael addition of polyamines at the double bond of maleimide terminal groups and further reaction of the secondary amine formed. However, products which are soluble or homogeneously dispersible in water are particularly preferred. Crosslinking products are not preferred as leather auxiliaries for the use according to the invention. In the case of branched structures, preferred copolymers are those which have a proportion of <5 mol % (based on structural units I) of branched structures, in order to ensure sufficient solubility or miscibility with water. However, it may be possible to reduce the molar masses by the concomitant use of monofunctional compounds in the condensation reaction so that a higher proportion of branches is also obtainable. Suitable monofunctional compounds for regulating the molar masses are monocarboxylic acids, such as, for example, formic acid, acetic acid, propionic acid, benzoic acid, cyclohexanecarboxylic acid or stearic acid, or monoamines, such as, for example, butylamine, dibutylamine, aminoethanol, hexylamine, dodecylamine, hexadecylamine, octadecylamine or octadecenylamine, and the abovementioned monofunctional polyetheramines.

According to the preferred embodiment A 1, a particularly preferred process for the preparation of the polyether-modified polymers is one which is characterized in that maleic acid or maleic anhydride A-1)—or optionally mixtures thereof with other dicarboxylic acids, tricarboxylic acids or anhydrides and/or tetracarboxylic acids or bisanhydrides from the group consisting of building blocks which lead to trivalent radicals W in the formula (also process component A-1)—is or are reacted in the presence of a solvent, such as diethylene glycol, ethylene glycol, propylene glycol, N-methylpyrrolidone or pyrrolidone, with ammonia and a diamino-functional polyether B-2) having an average molecular weight of 200 to 6 000 g/mol, simultaneously or in succession, in the temperature range between 5° C. and 100° C., the reaction mixture obtained is condensed with elimination of water in vacuo at temperatures between 80° C. and 200° C., preferably 80° C. to 160° C., and the polyether-modified polysuccinimide obtained is reacted in a solvent with primary amines having 8 to 30 carbon atoms D-1) and optionally monofunctional polyetheramines D-2) which preferably have an average molar mass of 600 to 4 000 g/mol, at above 120° C., preferably 120° C. to 170° C., and then dispersed in the presence of a base, preferably a water-soluble amine, in particular an alkanolamine, and/or sodium hydroxide solution, in water at temperatures of 70° C. to 140° C. (process step E), so that all succinimide groups still present react with ring opening, the pH is adjusted to values of 7 to 11 with a base, the product is subjected to bleaching with hydrogen peroxide at a temperature of 30° C. to 90° C. and any hydrogen peroxide radicals still present are treated with a reducing agent, in particular an enzyme formulation, which is suitable for the degradation of hydrogen peroxide radicals, preferably a catalase formulation.

In a very particularly preferred embodiment A1, maleic acid or maleic anhydride is reacted with ammonia and the diamines B-2) containing polyether groups (referred to below in short as “polyetherdiamine”) and the reaction mixture is then condensed with elimination of water. It is particularly advantageous first to react maleic acid (anhydride) with ammonia and with the polyetherdiamine B-2) in a temperature range between 5° C. and 100° C. and then to convert the reaction mixture thermally into a polycondensate containing polysuccinimide units. It is preferable to carry out the reaction with ammonia and with the polyether containing amino groups in succession, the sequence depending on whether a homogeneous reaction mixture is obtained. It may be advantageous first to carry out the reaction with the polyetherdiamine B-2) and to add a solvent in order to obtain a homogeneous reaction mixture and then to react the reaction product with aqueous ammonia. After the polycondensation, the copolymer contains predominantly succinimide structural units, amido groups and optionally still free carboxyl groups, which may be present in the form of the ammonium salts or of the polyetheramine salts. The resulting polycondensate containing polysuccinimide groups is preferably reacted with a C₁-C₆₀-alkylamine D-1), in particular a C₈-C₆₀-alkylamine, particularly preferably a C₁₂-C₃₀-alkylamine, optionally in the presence of a monofunctional polyetheramine D-2), and then dispersed in water in the presence of a base. Thereafter, after pH adjustment to a pH value between 6 and 10, bleaching is effected with hydrogen peroxide and excess oxidizing agent is removed by means of a reducing agent.

This preparation process is shown in simplified form in the following reaction scheme and with reference to specific starting materials:

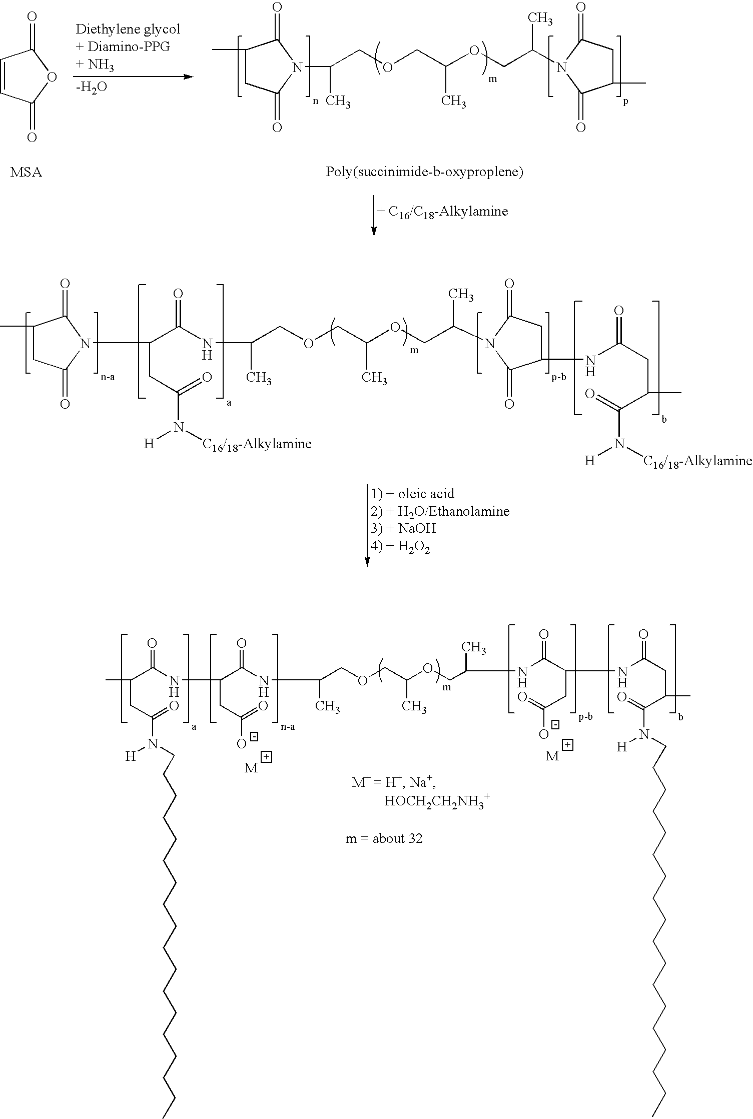

The reaction of polysuccinimide with amines is known in principle; cf. for example DE-A 2 253 190, EP-A 274 127, EP-A 406 623 and EP-A 519 119, U.S. Pat. No. 3,846,380, U.S. Pat. No. 3,927,204 and U.S. Pat. No. 4,363,797; P. Neri et al., Macromol. Syntheses 8, 25. The reaction of the polyether-modified polysuccinimides is preferably carried out in organic solvents. Suitable solvents are, for example, lactams, such as caprolactam, N-methylpyrrolidone, N-methylcaprolactam, polyalkylenediols and the mono- and diethers thereof, such as ethylene glycol, diethylene glycol, triethylene glycol, ethylene glycol dimethyl and diethyl ether and diethylene glycol monoethyl ether, and pyrrolidinone, N-methylpyrrolidinone, dimethylformamide and dimethyl sulphoxide. Preferred solvents are diethylene glycol, ethylene glycol and pyrrolidinone. The solvent content will as a rule not exceed 30% by weight, based on the total reaction mixture.

The reaction with amines HNR³R⁴ and optionally with monofunctional amino-polyethers HNR⁵R⁶ is carried out in a temperature range of 100° C. to 180° C., preferably of 120 to 150° C., the reaction times generally being from 3 to 24 hours, preferably 4 to 8 hours.

The polyether-modified (co)polymers according to the invention can be prepared from the polycondensate containing succinimide groups by opening of the remaining incorporated succinimide rings by means of bases and water. Suitable ring-opening bases are preferably alkali metal hydroxides, carbonates and bicarbonates, in particular sodium and potassium hydroxide and sodium carbonate, and ammonia and amines—including the amines serving as reactants.

According to a further embodiment A 2, a particularly preferred process for the preparation of the polyether-modified polymers is one which is characterized in that maleic acid or maleic anhydride A-1)—or optionally the mixtures thereof with other dicarboxylic acids, tricarboxylic acids or anhydrides and/or tetracarboxylic acids or bisanhydrides from the group consisting of building blocks which lead to the trivalent radicals W in the formula (also process component A-1)—is or are reacted with aqueous ammonia solution, preferably in the molar ratio of sum of the components A-1) to ammonia=1:0.5 to 1:2, and preferably in the presence of the abovementioned solvents, and condensation is then effected with distilling off of water in vacuo at temperatures between 80° C. and 200° C., preferably of 80° C. to 160° C., the resulting polycondensate containing polysuccinimide groups is reacted with the amines HNR³R⁴ and monofunctional aminopolyethers HNR⁵R⁶ at above 120° C., preferably from 120° C. to 180° C., preferably 120° C. to 160° C., and the reaction mixture obtained is then reacted with ring-opening base with simultaneous dispersing in water. For the reaction with the amines HNR³R⁴ and monofunctional aminopolyethers HNR⁵R⁶, a reaction time of 3 to 24, preferably of 4 to 8, hours is as a rule sufficient. The concomitant use of an organic solvent is advantageous in order for the mixture to remain stirrable. As a result of the reaction of the remaining succinimide rings in the polycondensate with ring-opening base and water, the polyether-modified copolymers according to the invention are obtained. The ring opening by simultaneous addition of water and base to the melt or to the solution of the polymer at 70° C. to 140° C. is preferred. Thereafter, after pH adjustment to a pH value between 6 and 10, bleaching is effected with hydrogen peroxide and excess oxidizing agent is removed by means of a reducing agent.

The invention furthermore relates to the (co)polymers obtainable by the processes according to the invention.

The polyether-modified polymers according to the invention, in particular polyaspartamides, are preferably self-dispersing. However, external dispersants may also be used; suitable external dispersants are in principle cationic, anionic and nonionic dispersants, as described, for example, in “Methoden der organischen Chemie” [Methods of Organic Chemistry] (Houben-Weyl), 4th Edition, Volume XIV/1, Georg Thieme Verlag, Stuttgart 1961, page 190 et seq.

Preferred dispersants include, for example, C₈-C₁₈-n-alkyl sulphates, C₈-C₁₈-n-alkyl benzenesulphonates, C₈-C₁₈-n-alkyl-trimethyl-ammonium salts, n-di-C₈-C₁₈-alkyl-dimethyl-ammonium salts, C₈-C₁₈-n-alkyl carboxylates, C₈-C₁₈-n-alkyl-dimethylamine oxides, C₈-C₁₈-n-alkyl-dimethylphosphine oxides and—preferably—oligoethylene glycol mono-C₆-C₁₈-alkyl ethers having on average 2 to 30 ethoxy groups per molecule. The n-alkyl radicals may also be replaced by partially unsaturated linear aliphatic radicals. Particularly preferred dispersants are oligoethylene glycol mono-C₁₀-C₁₄-alkyl ethers having on average 4 to 12 ethoxy groups per molecule, in particular oligoethylene glycol mono-C₁₂-alkyl ethers having on average 8 ethoxy groups per molecule.

Preferred dispersants furthermore include oleic acid, oleic acid sarcosides, ricinoleic acid, stearic acid, fatty acid partial esters of polyols, such as glycerol, trimethylolpropane or pentaerythritol, and the acylation, ethoxylation and propoxylation products thereof, e.g. glyceryl monostearate and monooleate, sorbitan monostearate and monooleate, sorbitan tristearate and trioleate, and the reaction products thereof with dicarboxylic anhydrides, such as succinic anhydride, maleic anhydride, phthalic anhydride or tetrahydrophthalic anhydride, reaction products of bis-(hydroxymethyl)-tricycoldecane and maleic anhydride or succinic anhydride and the derivatives thereof, preferably in the form of their alkali metal or ammonium salts.

Particularly preferred dispersants are salts of long-chain fatty acids, preferably of oleic acid, and an amino alcohol, preferably hydroxyethylamine, bishydroxyethylamine or trishydroxyethylarmine.

For the preparation of an aqueous dispersion, it is generally advisable to meter the dispersant into the reaction mixture before, during or after the dispersing, with stirring at temperatures of 70° C. to 140° C. It is also possible to disperse the reaction mixture in an aqueous dispersant solution.

The dispersant content is in general not more than 30, preferably 3 to 15, % by weight, based on prepared dispersion.

The solids content of the dispersions is preferably 5 to 60% by weight, particularly preferably 10 to 40% by weight. The mean particle size of the dispersed polyaspartamides is in general 50 to 1 000, preferably 50 to 700 and in particular 80 to 400 nm. The pH during the dispersing is preferably adjusted to between 5 and 11, particularly preferably 6 to 10.

In order to facilitate the penetration of the auxiliaries into the leather, it may be desirable to reduce the particle size of the disperse phase. For this purpose, a preemulsion already obtained can be aftertreated under a high shear gradient in known dispersing machines, such as jet dispersers having suitable nozzles, high-pressure emulsifying machines or mixers based on the rotor-stator principle. It is also possible to produce the dispersion in situ in the chambers or nozzles of the apparatuses mentioned. The duration of dispersing may be from a few minutes to 4 hours. The dispersing is preferably carried out in a temperature range between 20 and 75° C.

The invention therefore furthermore relates to aqueous dispersions containing the polymer according to the invention, preferably having solids contents of 10 to 60% by weight. The dispersions having a solids content below 35% by weight are generally present in the form of low-viscosity emulsions. The pH of the dispersions is preferably 4.5 to 12, preferably in the pH range from 4.5 to 10, in particular 5 to 8.

In the context of the invention, it is furthermore advantageous to subject the dispersions obtained to bleaching. The bleaching can be carried out oxidatively or reductively. Oxidative bleaching is preferred. A suitable oxidizing agent is hydrogen peroxide or alkali metal or ammonium persulphate in aqueous solution. The bleaching can be carried out in the temperature range between 20 and 90° C., preferably between 30 and 60° C. Unconsumed bleach is then destroyed using a reducing agent. Suitable reducing agents are, for example, sodium hydrogen sulphite solution or peroxide-decomposing enzyme formulations, such as BAYLASE® EPK (product of BAYER AG). The decomposition of residues of the oxidizing agent is advantageously carried out at temperatures between 20 and 45° C. in the pH range between 5 and 8.

The invention furthermore relates to the use of the polymer according to the invention for the treatment of leather and the precursors thereof from the leather production processes. “Precursors from the leather production processes” in the context of this definition are, for example, raw hides, pelt material, wet white, wet blue and all substrates which are present during the retanning, dyeing and fatliquoring in the tanning drum up to the finished crust. Preferred substrates are wet blue or wet white. The treatment of wet white and wet blue in all phases of the leather production process up to the crust leather is preferred. The (co)polymers according to the invention are advantageously used in particular during or after tanning with mineral tanning agents, vegetable tanning agents and syntans. Wet white is to be understood as meaning leathers which were obtained by means of chromium-free tanning methods and have a shrinkage temperature of more than 60° C. and are processible on shaving machines. The following may be mentioned as tanning agents in this case: titanium, zirconium, iron and aluminium tanning agents, aldehydes, isocyanates, blocked isocyanates, epoxides, oxazolidines, etc.

In the treatment with the compounds according to the invention, the leather is preferably brought into contact with an aqueous liquor which contains these compounds, by application by means of rolls or in a container, preferably in a tanning drum. After the treatment, the leather is generally acidified and dried.

The individual process steps are to be illustrated—without restricting the usability in the case of other substrates—by wet blue (chrome-tanned hides) by way of example:
1. Neutralization of the wet blue material
2. Washing
3. Addition of the liquor containing the polyether-modified polymer according to the invention, and treatment up to complete exhaustion of the liquor. The liquor may contain further customary products, such as tanning agents, retanning agents, dyes, fatliquoring agents, water repellents and auxiliaries, depending on the desired leather article.
4. Reduction of the pH by the addition of a carboxylic acid to pH values of <4.5, preferably to 3.0 to 4.5
5. Washing
6. Drying.

In a particular embodiment, leather can also be treated subsequently with a dispersion which contains the polyether-modified polymers to be used according to the invention.

Other auxiliaries can be concomitantly used in the liquor which contains the polymer according to the invention or separately therefrom. These include, for example: polymeric retanning agents based on known polyacrylate dispersions, leather dyes, vegetable tanning agents, syntans, fatliquoring agents, neutral oils and water repellents. It is possible to use only those combinations which are compatible with the dispersions of the polyether-modified polymers according to the invention. The use of additives having an anionic charge is as a rule unproblematic at a liquor pH of 6 to 4.5. These additives can, however, be particularly advantageously used before or after the use of the polymers to be used according to the invention.

Suitable vegetable tanning agents are, for example, chestnut extracts, mimosa, quebracho, etc.

Suitable syntans are, for example, synthetic organic tanning agents known to a person skilled in the art (cf. K. Faber, “Gerbmittel, Gerbung und Nachgerbung” [Tanning Agents, Tanning and Retanning], Frankfurt 1984).

Suitable fatliquoring agents are, for example, oxidized and/or sulphoxidized, preferably halogen-free C₁₆₋₃₀-hydrocarbons and/or C₃₂₋₁₀₀-waxes, C₂₋₅₄-polycarboxylic acids partly esterified with C₈₋₃₀-alcohols, such as octadecyl citrate, glutaric acid monooleate, C₂₋₅₄-polyols at least partly esterified with C₁₂₋₂₄-fatty acids, such as sorbitan, glyceryl, trimethylolpropane and pentaerythrityl monostearate, monooleate, distearate, dioleate, monolaurate, etc.

Suitable neutral oils are, for example, animal and/or vegetable fats and oils, such as neatsfoot oil, fish oil, sunflower oil, rapeseed oil, coconut oil, palm kernel oil, soya oil, preferably nonhalogenated fatty esters, methyl oleate, liquid paraffin and tallow.

Suitable water repellents are silicone emulsions, and polymer dispersions containing perfluoroalkyl groups, including polyurethanes containing perfluoroalkyl groups. Here, reference may be made to the literature (cf. M. Hollstein “Entfetten, Fetten und Hydrophobieren bei der Lederherstellung” [Degreasing, Fatliquoring and Imparting Water Repellency in Leather Production], Frankfurt, 1988; Ullmann's Encyclopädie der technischen Chemie [Ulmann's Encyclopaedia of Industrial Chemistry], key word leather).

The polymers according to the invention also give a pleasantly soft hand without further auxiliaries of the abovementioned categories. Moreover, it is surprising that the tinctorial properties and the levelness are improved and at the same time the leathers have very good fullness.

A reduction in the pH by addition of carboxylic acid is sufficient for fixing the polyether-modified polymers according to the invention. The liquor exhaustion is as a rule so good that addition of the acid to the drum is possible even without discharging the liquor. Preferred pH values should be less than 4.5 in order to achieve sufficient fixing in the leather. The range from 3.0 to 4.5 is particularly preferred.

Instead of fixing with a carboxylic acid, fixing can also be effected by adding polyvalent metal compounds, such as aluminium, titanium, zirconium or chromium salts or carboxylates or alkoxides, in the liquor.

Up to 15% by weight, preferably up to 7% by weight, of active substance, based on shaved weight, are preferably used according to the invention for achieving an optimum effect. The pH of the liquor at the beginning of the treatment is preferably 5 to 7. The pH can optionally be corrected by adding bases, such as ammonia. The treatment is preferably effected at a temperature between 20 and 80° C., preferably at 35 to 60° C. After the treatment, a pH of about 4 to 6 generally results in the liquor. For example, the pH can be reduced to 3 to 4.5 by adding a carboxylic acid. A preferred carboxylic acid for the acidification is formic acid.

The use, according to the invention, of the polyether-modified polymers gives very soft leathers having a level dyeing and particularly good fullness and reduced water absorption. If there are particular requirements with regard to the plasticizing effect, additional polymeric fatliquoring agents, for example fat-modified aminocarboxylic acid derivatives (for example LEVOTAN® L, commercial product of Bayer AG) or hydrophobically modified polyacrylate dispersions (for example Lubritan® WP, commercial product of Rohm and Haas), can be used. Another preferred polyacrylate-based fatliquoring agent is XERODERM® P-AF (commercial product of BAYER AG).

In the case of higher requirements with respect to the water repellency effect, it is possible to carry out a final step for imparting water repellency using silicone active substances or fluorocarbon resins. These active substances are known to a person skilled in the art and can be metered into the same drum after a liquor run time of, preferably, 30 minutes. Usually, anionic active substances are preferred since they are very compatible with the dispersions of the polyether-modified polyaspartamides to be used according to the invention. Particularly good results with regard to the water repellency effect are obtained, for example, with silicone emulsions (for example XERODERM® S-AF, product of BAYER AG). However, it is also possible to combine other commercial silicone emulsions with the products according to the invention.

The invention furthermore relates to leathers containing the polymers according to the invention.

The stated percentages in the examples below are based in each case on weight, unless stated otherwise; parts are parts by weight.

### PREPARATION EXAMPLES

Description of specific raw materials

| | |
|---|---|
| RESPUMIT ® S | antifoam, silicone-containing |
| | emulsion, Bayer AG |
| BAYLASE ® EPK | enzymatic reducing agent, BAYER AG |
| Armeen ® HT | hydrogenated tallow fatty amine; |
| | C₁₆/C₁₈-alkylamine mixture having |
| | primary amino groups; from Akzo |
| Polyether 1 | JEFFAMINE ® D-2000, from Huntsman |
| | α,ω-bis(2-aminopropyl)poly- |
| | (oxypropylene), |
| | average molar mass about 2000 g/mol, |
| | N content = 1.41% by weight |
| Polyether 2 | JEFFAMINE ® XTJ 506 (M-1000), |
| | from Huntsman |
| | α-methyl-ω-(2-aminopropyl)- |
| | poly(oxypropylene-co-oxyethylene) |
| | average molar mass about 1000 g/mol, |
| | EO/PO = 19/3, N content = 1.36% |
| | by weight |
| Polyether 3 | JEFFAMINE ® XTJ 505 (M-600), |
| | from Huntsman |
| | α-methyl-ω-(2-aminopropyl)- |
| | poly(oxypropylene-co-oxyethylene) |
| | average molar mass about 600 g/mol, |
| | EO/PO = 1/9, N content = 2.31% |
| | by weight |
| Polyether 4 | JEFFAMINE ® XTJ 507 (M-2005), |
| | from Huntsman |
| | α-methyl-ω-(2-aminopropyl)- |
| | poly(oxypropylene-co-oxyethylene) |
| | average molar mass about 2000 g/mol, |
| | EO/PO = 6/39, N content = 0.69% |
| | by weight |

Auxiliaries used in the use examples:

| | |
|---|---|
| TANIGAN ® WLF | condensate of aromatic sulphonic |
| | acids; synthetic tanning agent, |
| | Bayer AG |
| TANIGAN ® BN | condensate of aromatic sulphonic |
| | acids; synthetic tanning agent, |
| | Bayer AG; |
| BAYKANOL ® SL | condensate of aromatic sulphonic |
| | acids; synthetic dyeing auxiliary, |
| | Bayer AG; |
| RETINGAN ® RD liquid | formaldehyde condensate; |
| | resin tanning agent, Bayer AG; |
| Lubritan ® GX | polymer retanning agent having |
| | plasticizing properties, Rohm |
| | and Haas; |
| BAYKANOL ® Licker CAR | synthetic fatliquoring agent, |
| | product of Bayer AG; |
| BAYGENAL ® Grey L-NG | dye; Bayer AG |
| TANIGAN ® AN | condensate of aromatic sulphonic |
| | acids and ligninsulphonate; |
| | synthetic tanning agent, Bayer AG; |
| TANIGAN ® PAK-S | condensate of aromatic sulphonic |
| | acids; synthetic tanning auxiliary, |
| | Bayer AG; |
| RETINGAN ® R 7 | formaldehyde condensate with |
| | dicyandiamide; resin tanning agent, |
| | Bayer AG; |
| LEVOTAN ® C | polymeric retanning agent, |
| | Bayer-AG; |
| BAYKANOL ® Licker PAN | plasticizing fatliquoring agent |
| | based on marine oils; Bayer AG; |
| BAYKANOL ® Licker NB | synthetic fatliquoring agent; |
| | Bayer AG; |
| BAYGENAL ® Brown CGG | dye; Bayer AG; |
| BAYGENAL ® | red-brown L-N dye; Bayer AG; |
| TANIGAN ® F | condensate of aromatic sulphonic |
| | acids and ligninsulphonate; |
| | synthetic tanning agent, Bayer AG; |
| TANIGAN ® OS | condensate of aromatic sulphonic |
| | acids; synthetic tanning agent, |
| | Bayer AG; |
| XERODERM ® P-AF | polymeric fatliquoring agent, |
| | Bayer-AG; |
| XERODERM ® S-AF | silicone-containing water |
| | repellent; Bayer AG; |

### Example 1

Molar Ratio Maleic Anhydride:Polyether 1:NH₃:Fatty Amine=1:0.1:1:0.25

195.0 g of diethylene glycol are initially introduced into a stirred reactor. 245.1 g of maleic anhydride (2.5 mol) are introduced at room temperature with stirring, and the mixture is heated to 65° C. until the exothermic reaction begins. 500.0 g (0.25 mol) of polyether 1 are then metered in while cooling, so that the temperature remains in the range from 50 to 70° C. Thereafter, 170.0 g (2.5 mol) of 25% ammonia water are metered in in the course of 2.5 hours while cooling at between 50 and 60° C. Stirring is continued for 1 hour at 70° C.

The reactor is then evacuated stepwise to 200 mbar and further heated with simultaneous distillation of water until a bottom temperature of 130-140° C. is reached and no more water of reaction passes over.

168.5 g (0.625 mol) of Armeen HT, which was melted beforehand at 80° C., are added at 140° C. under nitrogen. Stirring is effected for 7 hours at 135-140° C. Thereafter, 42.0 g of oleic acid are added, stirring is continued for 15 minutes and a solution of 2000 g of water and 18.0 g of ethanolamine is then metered in in the course of 90 minutes, beginning at 125° C. and at maximum stirring speed. After the start of the feed of the ethanolamine solution, 80 g of 50% sodium hydroxide solution are added simultaneously in 10 minutes. A brown dispersion is obtained. During the dispersing, the internal temperature decreases to 85° C. After cooling to 65° C., 0.5 g of an antifoam (RESPUMIT S) is added. With a reduced stirring speed, 100.0 g of 35% hydrogen peroxide are metered in in the course of 20 minutes at 60-70° C. Stirring is effected for 4 hours at 65° C. and cooling to 30-38° C. is then effected. At 30-38° C., 1.0 g of RESPUMIT S and then 3.0 g of BAYLASE EPK are added in 2 portions. Stirring is continued for 3 hours at 35° C., cooling to below 30° C. is effected and filling is effected via a 100 μm filter.

| | |
|---|---|
| Solids content: | 30.7% by weight |
| Appearance: | yellow dispersion |
| pH: | 7.78 (diluted 1:4 (c = 20%), potentiometrically) |
| Viscosity (20°): | 40.3 seconds (Ford cup, 4 mm nozzle) |
| | 510 mPa · s at 20° C., D = 100 sec⁻¹ |
| Mean particle size: | d₅₀ = 140 nm (Coulter LS 230) |
| Mw (GPC) | 11200 g/mol |

Preparation of Precursor A for Examples 2 and 3:
Molar Ratio Maleic Anhydride:Ammonia:Fatty Amine=1:1.2:0.15

Precursor A is prepared analogously to the conditions of example 1:
Initially introduced material: 390.0 g of diethylene glycol
Feed 1: 490.3 g (5 mol) of maleic anhydride, then
Feed 2: 408.0 g (6 mol) of ammonia water (25%) in 3 hours at 50-60° C.

Stirring is then continued for 1 hour at 70° C. Thereafter, vacuum distillation (max. 200 mbar) is effected until the bottom temperature has reached 140° C. and no more water distils off. At 140° C., feed 3 is then carried out: 202.2 g (0.75 mol) of Armeen HT which was melted before at 80° C. The batch is stirred for 3 hours at 140° C. 1 094.5 g of a red-brown melt is obtained and said melt is discharged and is comminuted after cooling. Equivalent weight, based on monomer unit maleic anhydride=218.9 g (1 mol of maleic anhydride corresponds to 218.9 g of precursor A).

### Example 2

Molar Ratio Maleic Anhydride:NH₃:Fatty Amine:Polyether 2=1:1.2:0.15:0.35

109.45 g of the precursor A described above (0.5 mol) are initially introduced at 140° C. 175 g (0.175 mol) of a melt of polyether 2, thermostated at 80° C., are then added. The batch is stirred for a further 4 hours at 140° C. and then cooled to 115° C. Thereafter, 14.25 g of oleic acid are added, stirring is effected for 5 minutes and a solution of 465 g of water and 6.1 g of ethanolamine is metered in in the course of 60 minutes, beginning at 115° C. and at maximum stirrer speed, the batch being cooled to 65° C. After addition of 0.5 g of RESPUMIT S, 20.0 g of 35% hydrogen peroxide are added in the course of 10 minutes at 65° C. Stirring is continued for 4 hours at 65° C. and cooling to 35° C. is then effected. At 35° C., 0.2 g of RESPUMIT S and 1.5 g of BAYLASE EPK are metered in in 2 portions in the course of 30 minutes. After the end of the addition, stirring is continued for 3 hours at 25-35° C. and filling via a 100 μm filter is then effected.

| | |
|---|---|
| Solids content: | 34.6% by weight |
| Appearance: | pale brown solution |
| pH: | 6.23 (diluted 1:4 (c = 20%), potentiometrically) |
| Viscosity (20°): | 16 seconds (Ford cup, 4 mm nozzle), |
| | 59 mPa · s (100 s⁻¹, 20° C.) |

### Example 3

Molar Ratio Maleic Anhydride:NH₃:Fatty Amine:Polyether 3=1:1.2:0.15:0.15

Analogously to example 2, 109.45 g of precursor A (0.5 mol) are reacted with 45 g (0.075 mol) of polyether 3 at 140° C. for 4 hours. After cooling to 65° C., 8.4 g of oleic acid are added and a solution of 280 g of water and 3.6 g of ethanolamine is metered in in the course of 60 minutes. Stirring is continued for 10 minutes. 20.0 g of 35% hydrogen peroxide are then added at 65° C. in the course of 10 minutes. The batch is stirred for 3 hours at 65° C. At 35° C., 0.2 g of RESPUMIT S and then 1.5 g of BAYLASE EPK are metered in in 2 portions in the course of 30 minutes. After the end of the addition, stirring is continued for 2 hours at 25-35° C. and the batch is then filled via a 100 μm filter.

| | |
|---|---|
| Solids content: | 28.4% by weight |
| Appearance: | yellow-orange dispersion |
| pH: | 5.19 (diluted 1:4 (c = 20%), potentiometrically) |
| Viscosity: | about 80 mPa · s (100 s⁻¹, 20° C.) |

Preparation of Precursor B for Examples 4 to 6:
Molar Ratio Maleic Anhydride:Ammonia:Fatty Amine=1:1.2:0.25

The precursor B is prepared analogously to the precursor A, except that 337.0 g (1.25 mol) of Armeen HT, which was melted beforehand at 80° C., are used. The reaction time is 3 hours at 140° C. 1229.3 g of red-brown melt are discharged and, after cooling, are comminuted.

Equivalent weight of precursor B, based on monomer unit maleic anhydride=245.86 g (1 mol of maleic anhydride=245.86 g of precursor B).

### Example 4

Molar Ratio Maleic Anhydride:NH₃:Fatty Amine:Polyether 2=1:1.2:0.25:0.15

75 g (0.075 mol) of a melt of polyether 2, thermostated at 80° C., are metered into 122.9 g of the precursor B described above (0.5 mol) at 140° C., and the reaction is effected for 4 hours at 140° C. Thereafter, 8.4 g of oleic acid are added at 115° C., stirring is effected for 5 minutes and 365 g of water and 3.63 g of ethanolamine are metered in in the course of 60 minutes, beginning at 115° C. and at maximum stirring speed, the batch being cooled to 65° C. After addition of 0.5 g of RESPUMIT S, 20.0 g of 35% hydrogen peroxide are added in the course of 10 minutes at 65° C. Stirring is continued for 4 hours at 65° C., and the reaction mixture is cooled to 35° C. At 35° C., 0.25 g of RESPUMIT S and then 1.5 g of BAYLASE EPK are metered in in 2 portions in the course of 15 minutes. After the end of the addition, stirring is continued for 3 hours at 35° C. and the batch is then filled via a 100 μm filter.

| | |
|---|---|
| Solids content: | 29.6% by weight |
| Appearance: | pale yellow dispersion |
| pH: | 5.58 (diluted 1:4 (c = 20%), potentiometrically) |
| Viscosity (20°): | about 120 mPa · s (100 s⁻¹, 20° C.) |

### Example 5

Molar Ratio Maleic Anhydride:NH₃:Fatty Amine:Polyether 2:Primary/Tertiary Amine=1:1.2:0.25:0.25:0.25

125 g (0.125 mol) of polyether 2 and 12.8 g (0.125 mol) of 3-aminopropyl-dimethylamine are metered into 122.9 g of precursor B (0.5 mol) analogously to example 4, and the mixture is stirred for 6 hours at 140° C. Thereafter, 8.4 g of maleic acid are added at 125° C., stirring is effected for 5 minutes and a solution of 465 g of water and 3.63 g of ethanolamine are metered in in the course of 60 minutes, beginning at 125° C. and at maximum stirring speed, and stirring is continued for a further 30 minutes, the batch being cooled to 65° C. At 65° C., 20.0 g of 35% hydrogen peroxide are added in the course of 10 minutes. Stirring is continued for 4 hours at 65° C. Thereafter, 0.25 g of RESPUMIT S and then 1.5 g of BAYLASE EPK are metered in in 2 portions at 35° C. in the course of 15 minutes. After the end of the addition, stirring is continued for 3 hours at 35° C. and the batch is then filled via a 100 μm filter.

| | |
|---|---|
| Solids content: | 30.4% by weight |
| Appearance: | brown, low-viscosity dispersion |
| pH: | 7.09 (diluted 1:4 (c = 20%), potentiometrically) |
| Viscosity (20°): | about 30 mPa · s (100 s⁻¹, 20° C.) |

### Example 6

Molar Ratio Maleic Anhydride:NH₃:Fatty Amine:Polyether 4=1:1.2:0.25:0.225

225 g (0.1125 mol) of polyether 4 are metered into 122.9 g of precursor B (0.5 mol) analogously to example 4 at 140° C. and the reaction is carried out for 7 hours at 140° C. Thereafter, cooling to 120° C. is effected, 16.8 g of oleic acid are added, stirring is effected for 15 minutes and a solution of 620 g of water and 7.26 g of ethanolamine is then metered in in the course of 60 minutes, beginning at 115° C. and at maximum stirrer speed, the batch being cooled to 65° C. Thereafter, stirring is continued for 30 minutes and 3 g of 50% sodium hydroxide in 50 g of water are added. At 65° C., 20.0 g of 35% hydrogen peroxide are added in the course of 10 minutes. Stirring is continued for 3 hours at 65° C. 100 g of water are then added. At 35° C., 0.25 g of RESPUMIT S and then 1.5 g of BAYLASE EPK are metered in in the course of 15 minutes. After the end of the addition, stirring is continued for 3 hours at 25-35° C. and the batch is then filled via a 100 μm filter.

| | |
|---|---|
| Solids content: | 28.8% by weight |
| Appearance: | viscous yellow dispersion |
| pH: | 7.24 (diluted 1:4 (c = 20%), potentiometrically) |
| Mean particle size: | 119 nm |

Preparation of Precursor C for Example 7:
Molar Ratio Maleic Anhydride/Trimellitic Anhydride:NH₃:Fatty Amine=0.83/0.17:1.25: 0.208

234.0 g of diethylene glycol are initially introduced into a reaction vessel, and 294.15 g of maleic anhydride (3 mol) and 115.2 g of trimellitic anhydride (0.6 mol) are introduced at room temperature with stirring. The mixture is heated to 65° C., an exothermic reaction beginning at 50° C. and the temperature increasing to 87° C. After cooling to 55° C., 306.0 g of ammonia water (25%, 4.5 mol) are metered in in 3 hours at 50-60° C. After the end of the addition, stirring is continued for 1 hour at 70° C. In the subsequent vacuum distillation (200 mbar), heating is effected until the bottom temperature has reached 140° C. and no more distillate passes over.

202.2 g (0.75 mol) of a melt of Armeen HT at 80° C. are added under nitrogen at 140° C. The batch is stirred for 7.5 hours at 140° C. 872.8 g of red-brown melt are obtained, which melt is discharged and, after cooling, is comminuted.

Equivalent weight of precursor C, based on monomer unit maleic anhydride=290.9 g. (1 mol of maleic anhydride=290.9 g of precursor C).

### Example 7

Molar Ratio Maleic Anhydride/Trimellitic Anhydride:NH₃:Fatty Amine:Polyether 2=0.83/0.17:1.25:0.208:0.1245

145.15 g of the precursor C described above (0.5 mol) are melted in a reactor. 75 g (0.075 mol) of polyether 2, which was melted beforehand at 80° C., are then added at 140° C. The batch is stirred for 4 hours at 140° C. At 115° C., 8.4 g of oleic acid are added, stirring is effected for 5 minutes and a solution of 200 g of water and 3.65 g of ethanolamine is metered in in the course of 45 minutes, beginning at 115° C. and at maximum stirrer speed, the batch being cooled to 90° C. After addition of 0.15 g of RESPUMIT S, stirring is continued for 20 minutes and 7.5 g of 50% sodium hydroxide solution are added. Cooling to 65° C. is effected. At 65° C., 11.8 g of 35% hydrogen peroxide are added in the course of 10 minutes and stirring is continued for 3 hours. After cooling to 35° C., 0.25 g of RESPUMIT S and then 1.5 g of BAYLASE EPK are metered in in 15 minutes. Stirring is continued for a further 1 hour at 25-35° C. and the solids content is adjusted.

| | |
|---|---|
| Solids content: | 33.7% by weight |
| Appearance: | pale yellow dispersion |
| pH: | 5.62 (diluted 1:4 (c = 20%), potentiometrically) |
| Viscosity (20°): | 148 mPa · s (100 s⁻¹, 20° C.) |
| Mean particle size: | 292 nm |

### Example 8

Molar Ratio Maleic Anhydride:Polyether 1:NH₃:Fatty Amine:Polyether 2=1:0.05:1.0: 0.25:0.5

39.0 g of diethylene glycol are initially introduced into a stirred reactor. 49.05 g of maleic anhydride (0.5 mol) are introduced at room temperature with stirring, and the mixture is heated to 65° C. until the exothermic reaction begins. 50.0 g (0.025 mol) of polyether 1 are then metered in at 55° C. in 30 minutes while cooling. 34.0 g (0.5 mol) of 25% ammonia water are then metered in in the course of 45 minutes while cooling at between 50 and 60° C. Stirring is continued for 1 hour at 70° C. The reactor is then evacuated stepwise to 200 mbar and further heated with simultaneous distillation of water until a bottom temperature of 130-140° C. is reached and no more water of reaction passes over. 36 g of distillate are obtained. 33.7 g (0.125 mol) of Armeen HT which was melted beforehand at 80° C., are added at 140° C. Stirring is effected for 3 hours at 135-140° C. Thereafter, 250 g (0.25 mol) of polyether 2 (melt at 80° C.) are added and stirring is effected for 4 hours at 140° C. After cooling to 115° C., 8.4 g of oleic acid are added, stirring is continued for 10 minutes and a solution of 600 g of water and 3.6 g of ethanolamine is then metered in in the course of 60 minutes, beginning at 115° C. and at maximum stirring speed. After cooling to 65° C., 40.0 g of 35% hydrogen peroxide are metered in in the course of 10 minutes at 65° C. at reduced stirring speed. Stirring is effected for 3 hours at 65° C. and cooling to 35° C. is then effected, and 0.5 g of RESPUMIT S and 3.0 g of BAYLASE EPK are added in 2 portions. Stirring is continued for 3 hours at 35° C., cooling to below 30° C. is effected and filling is effected via a 100 μm filter.

| | |
|---|---|
| Solids content: | 37.7% by weight |
| Appearance: | dark brown solution |
| pH: | 7.79 (diluted 1:4 (c = 20%), potentiometrically) |

Use Examples for Leather Treatment

The examples given are intended to illustrate the invention but in no way restrict it. All stated amounts are based in each case on shaved weight.

Use Example 1

Simplified retanning of chrome-tanned leather for comparative testing;

Starting material: wet blue, 1.6 mm

| | | | | Time | |
|---|---|---|---|---|---|
| Operation | % | Product | ° C. | min. | Remark |
| Washing | 200 | Water | 30 | 10 | Liquor |
| | | | | | discharged |
| Neutralization | 100 | Water | 30 | | |
| | 1.0 | Sodium formate | | | |
| | 1.0 | Sodium | | over- | pH 5.0; liquor |
| | | bicarbonate | | night | discharged |
| Retanning | 100 | Water | 50 | | |
| | 2.5 | Test product | | 180 | pH 4.8 |
| Fixing | 0.5 | 85% strength | | 60 | pH 4.2; liquor |
| | | formic acid | | | discharged |
| | | 1:10 | | | |

Rinse, set out, hang up to dry

The leathers obtained were assessed with regard to the visual and haptic properties: rating: 5 is the best and 1 is the poorest rating.

| Test product | Full- | Soft- | | | Level- | Smooth- |
|---|---|---|---|---|---|---|
| according to | ness | ness | Colour | Colour | ness | ness |
| Example 1 | 4 | 3.5 | 3 | green | 4 | 4 |
| Example 2 | 5 | 2.5 | 4 | neutral | 4.5 | 4 |
| Example 3 | 3 | 3 | 3 | yellow | 5 | 4 |
| Example 4 | 4.5 | 3 | 2.5 | green | 2 | 3 |
| Example 5 | 5 | 2.5 | 3.5 | green | 3 | 3 |
| Example 6 | 3 | 4 | 3 | green | 4 | 4.5 |
| Example 7 | 2 | 4.5 | 5 | yellow | 5 | 4.5 |
| Example 8 | 4.5 | 2.5 | 5 | neutral | 5 | 4 |

The outstanding tinctorial properties of the products are evident. Regarding the softness and fullness, in particular the products comprising polyether 1 and polyether 2 should be singled out. The incorporation of the polyethers in the main chain surprisingly gives the product with the most advantageous all round properties. The product properties can be adapted to the requirements for the desired leather article by suitable choice of the ratios of the components.

The results show that polyether-modified polyaspartamides improve the fullness, and pleasantly soft leathers having good tinctorial properties are obtained.

Use Example 2

Production of an upholstery leather, comparison of different products;

Starting material: cattle wet blue, 1.2-1.3 mm:

| | | | | | Time | |
|---|---|---|---|---|---|---|
| Operation | + | % | Product | | min | Remark |
| | | 300 | Water 35° C. | | | |
| | | 0.1 | Formic acid | 1:10 | 20 | pH: 3.8 |
| | | | | | | Liquor |
| | | | | | | discharged |
| Neutral- | | 100 | Water 35° C. | | | |
| ization | | | | | | |
| | | 2.0 | Sodium formate | | | |
| | | 1.0 | Sodium | | 30 | pH: 6.8 |
| | | | bicarbonate | | | |
| | | 4.0 | Test product | 1:4 | 90 | pH: 6.4 |
| | | | | | | Liquor |
| | | | | | | discharged |
| | | 300 | Water 30° C. | | 10 | |
| Retanning | | 100 | Water 30° C. | | | |
| | | 6.0 | BAYKANOL ® | 1:4 | | |
| | | | Licker CAR | | | |
| | | 6.0 | Test product | 1:4 | 30 | pH: 6.7 |
| | + | 4.0 | TANIGAN ® WLF | | | |
| | | 2.0 | TANIGAN ® BN | | | |
| | | 6.0 | RETINGAN ® | 1:3 | 30 | |
| | | | RD liquid | | | |
| Dyeing | + | 2.0 | BAYKANOL ® SL | | | |
| | | 3.0 | BAYGENAL ® | | 60 | |
| | | | Grey L-NG | | | |
| | + | 100 | Water 60° C. | | 10 | |
| | + | 1.5 | LUBRITAN ® GX | 1:4 | 30 | |
| | + | 1.0 | Formic acid | 1:10 | 15 | |
| | + | 1.0 | Formic acid | 1:10 | 45 | pH: 3.9 |
| | | | | | | Liquor |
| | | | | | | discharged |
| | | 200 | Water 50° C. | | 10 | Liquor |
| | | | | | | discharged |
| | | 200 | Water 20° C. | | 10 | Liquor |
| | | | | | | discharged |

The leathers comprising a test product according to example 1 (leather A), a test product according to example 1 of DE-A 195 28 782 (leather B) and without test product (leather C) were rated as follows with regard to their properties:
Tight-grained character/milled grain pattern: A/B comparatively good, C is substantially poorer than A/B. Softness: A is almost as soft as B. C is substantially harder than A/B. Fullness: A is substantially fuller than B, followed by C with substantially less fullness.
Levelness/depth of colour: A/B have a comparable depth of colour and levelness, and C is substantially paler.
Overall assessment: A/B receives the best rating with respect to softness, fullness and milled grain pattern, A being fuller and more compact than B but B being softer than A. C is much poorer than the other leathers.
   Use Example 3

Production of a soft, milled upper leather

Starting material: Cattle wet blue, 1.6-1.8 mm.

| | | | Time | |
|---|---|---|---|---|
| % | Product | Dil* | min | Remark |
| 150 | Water 45° C. | | | |
| 0.3 | Formic acid | 1:10 | 10 | pH: 3.3 |
| | | | | Liquor discharged |
| 100 | Water 45° C. | | | |
| 3.0 | LEVOTAN ® C | 1:3 | 30 | pH: 3.5 |
| 3.0 | BLANCOROL ® RC | | | |
| 2.0 | TANIGAN ® AN | | 45 | pH: 4.2 |
| 2.0 | TANIGAN ® PAK-S) | | | |
| 0.5 | Sodium bicarbonate) | | 60 | pH: 4.8 |
| | | | | Liquor discharged |
| 200 | Water 40° C. | | 10 | Liquor discharged |
| 80 | Water 40° C. | | | |
| 5.0 | Product according to | 1:4 | 15 | pH: 5.1 |
| | example 1 | | | |
| 1.0 | BAYKANOL ® Licker PAN) | | | |
| 1.0 | BAYKANOL ® Licker NB) | 1:4 | 30 | pH: 5.0 |
| 4.0 | TANIGAN ® BN) | | | |
| 3.0 | RETINGAN ® R7) | | | |
| 1.3 | BAYGENAL ® Brown CGG) | | | |
| 1.7 | BAYGENAL ® Red-brown | | 60 | |
| | L-N) | | | |
| 1.0 | Formic acid | 1:10 | 30 | pH: 4.0 |
| | | | | Liquor discharged |
| 200 | Water 50° C. | | 10 | Liquor discharged |
| 100 | Water 50° C. | | | |
| 8.0 | BAYKANOL ® Licker NB) | | | |
| 2.0 | BAYKANOL ® Licker PAN) | 1:4 | 45 | |
| 1.0 | Formic acid | 1:10 | 30 | pH: 3.5 |
| | | | | Liquor discharged |
| 200 | Water 50° C. | | | |
| 0.4 | BAYGENAL1 ® Brown CGG) | | | |
| 0.6 | BAYGENAL ® Red-brown | 1:20 | 20 | |
| | L-N) | | | |
| 1.0 | Formic acid | 1:10 | 30 | pH: 3.3 |
| | | | | Liquor discharged |

*Ratio of the dilution with water on addition to the liquor

After drying and milling, a soft, full and round pale-brown leather having excellent levelness and low water absorption is obtained.

### Use Example 4

Production of a waterproof upper leather

Starting material: cattle wet blue, 1.8-2.0 mm;

| % | Product | | Time | Remark |
|---|---|---|---|---|
| 200 | Water 40° C. | | | |
| 0.2 | Formic acid | 1:10 | 15 | pH: 3.5 |
| | | | | Liquor discharged |
| 100 | Water 40° C. | | | |
| 2.0 | TANIGAN ® PAK-S | | | |
| 1.0 | Sodium bicarbonate | | | |
| 2.0 | TANIGAN ® F | | 15 | pH: 7.3 |
| 2.0 | XERODERM ® P-AF | 1:4 | 90 | pH: 5.7 |
| | | | | Liquor discharged |
| 200 | Water 40° C. | | 10 | Liquor discharged |
| 50 | Water 40° C. | | | |
| 6.0 | XERODERM ® P-AF | 1:4 | 15 | pH: 6.0 |
| 4.0 | Product according to | 1:4 | 15 | pH: 6.3 |
| | example 1 | | | |
| 4.0 | TANIGAN ® OS | | | |
| 4.0 | TANIGAN ® F | | | |
| 3.0 | RETINGAN ® R7 | | 15 | |
| 1.3 | BAYGENAL ® Brown CGG | | | |
| 1.7 | BAYGENAL ® Red-brown | | 90 | |
| | L-N | | | |
| 150 | Water 50° C. | | | |
| 1.0 | Formic acid | 1:10 | 30 | pH: 4.2 |
| | | | | Liquor discharged |
| 200 | Water 30° C. | | 10 | Liquor discharged |
| 50 | Water 30° C. | | | |
| 1.8 | Ammonium bicarbonate | | 60 | pH: 7.3 |
| 4.0 | XERODERM ® S-AF | 1:4 | 60 | pH: 7.1 |
| 1.0 | Formic acid | 1:10 | 10 | |
| 1.0 | Formic acid | 1:10 | 10 | |
| 1.0 | Formic acid | 1:10 | 10 | |
| 150 | Water 50° C. | | | |
| 1.0 | Formic acid | 1:10 | 60 | Overnight |
| | | | | pH: 3.4; |
| | | | | liquor discharged |
| 300 | Water 50° C. | | 10 | Liquor discharged |
| 300 | Water 20° C. | | 10 | Liquor discharged |

After drying and staking, a red-brown leather having very good fullness, softness and excellent levelness is obtained. Testing for waterproof character gave:

| | |
|---|---|
| Bally penetrometer: | Water penetration time >8 h at 7-8% |
| | water absorption, |
| Maeser penetrometer: | >50000 flexes, 7% water absorption. |

1. (Co)polymers which contain
a) structural units of the general formula I 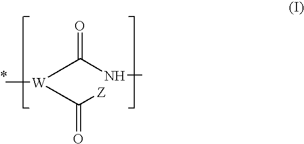
in which
W represents a trivalent radical from the group 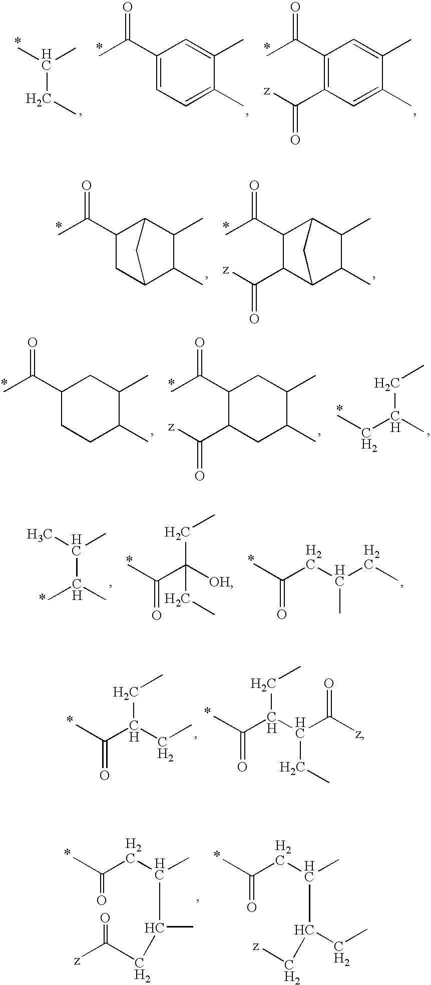
in which
* indicates the orientation for the incorporation of the radical W into the formula I, and
Z represents the radicals —OH, —O⁻M⁺ or —N—R¹R², in which R¹ and R², independently of one another, represent hydrogen, optionally substituted alkyl radicals, alkenyl radicals, aralkyl radicals or cycloalkyl radicals, which are optionally interrupted by O atoms, N atoms, Si atoms or amido, carbonate, urethane, urea, allophanate, biuret or isocyanurate groups or mixtures thereof, and
M⁺ represents H⁺ or an alkali metal ion, an NH₄ ion or a primary, secondary, tertiary or quaternary aliphatic ammonium radical
b) at least 10 mol %, based on the units of the formula I, of structural units of the general formula Ia 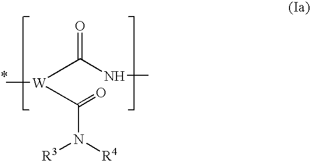
in which
R³ represents a hydrocarbon radical having C₁-C₆₀-atoms,
R⁴ represents hydrogen or has the same meaning as R³, and
c) polyether units having an average molecular weight of 200-6 000 g/mol.

2. (Co) polymers according to claim 1, characterized in that the formula I corresponds to the formula II 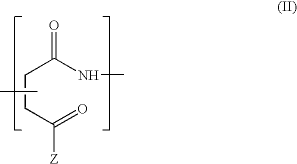
and the formula Ia corresponds to the formula Ib 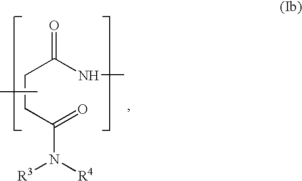
in which Z, R³ and R⁴ have the meaning stated in claim 1.

3. (Co)polymers according to claim 1, characterized in that the copolymer comprises more than 95% by weight, of structural units of the formula I and polyether units,
i) from 50 to 90 mol %, based on the sum of these two units, being accounted for by structural units of the formula I, of which at least 10 mol %, based on the units of the formula I, correspond to those of the formula Ia, and
ii) from 10 to 50 mol %, preferably 10 to 30 mol %, based on the sum of these two units, being accounted for by polyether units having an average molecular weight of 200 to 6 000 g/mol.

4. (Co)polymers according to claim 1, characterized in that the copolymer comprises more than 95% by weight, of structural units of the formula I, of which
i) at least 10 mol %, the structural units of the formula I, correspond to the formula Ia,
ii) at least 10 mol %, based on the units of the formula I, correspond to the formula Ic, 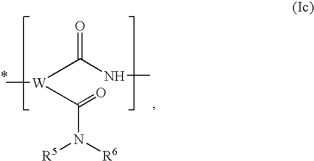
in which
W has the meaning stated in claim 1,
R⁵ represents a radical which contains more than two ether groups derived from C₂-C₆-alkylene oxide units and which is optionally interrupted by urethane, carbonate, urea, biuret, allophanate, isocyanate, alkylene, cycloalkylene or aralkylene groups and
R⁶ represents hydrogen or has the meaning of R⁵.

5. (Co)polymers according to claim 1, characterized in that the proportion of units where 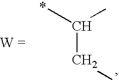
is more than 50 mol %, based on the sum of all units of the formula I.

6. Process for the preparation of the (co)polymers according to claim 1, comprising reacting
A-1) polycarboxylic acids or the anhydrides thereof selected from the group consisting of maleic acid, maleic anhydride, fumaric acid, itaconic acid, itaconic anhydride, citraconic acid, citraconic anhydride, 1,2,3-propanetricarboxylic acid, citric acid, 1,2,4-cyclohexanetricarboxylic acid, 1,2,3-cyclohexanetricarboxylic acid, 2,3,5-norbornanetricarboxylic acid, trimellitic acid, trimellitic anhydride, 1,2,3,4-butanetetracarboxylic acid, 1,2,4,5-cyclohexanetetracarboxylic acid, 2,3,5,6-norbornanetetracarboxylic acid, 2,3,5,6-norbornanetetracarboxylic anhydride, pyromellitic acid and pyromellitic bisanhydride
A-2) and optionally further polycarboxylic acids and/or anhydrides (V-2) differing from A-1) (for introduction of the bridge members V)
A-3) and optionally aminocarboxylic acids (V-3) or lactams (V-4) (for introducing the bridge members V) with
B-1) ammonia
B-2) and optionally with diamines which contain polyether groups and have an average molar mass of 200-6 000 g/mol
B-3) and optionally with additional polyamines (V-1 differing from B-2 (for introducing the bridge members V)
to give polycarboxamide-ammonium salts,
C) condensing the resultant of B) with elimination of water,
D-1) reacting the polycondensate obtained with amines of the formula HNR3R4, in which R³ and R⁴ have the meaning stated in claim 1,
D-2) and optionally with amines of the formula HNR5R6 which contain polyether groups and have a molar mass of 200-6 000 g/mol in which R⁵ represents a radical which contains more than two ether groups derived from C₂-C₆-alkylene oxide units and which is optionally interrupted by urethane, carbonate, urea, biuret, allophanate, isocyanate, alkylene, cycloalkylene or aralkylene groups and R⁶ represents hydrogen or has the meaning of R⁵, and
E) reacting the reaction product with a ring-opening base in the presence of water,
with the proviso that at least one of the two components B-2) or D-2) is used in the reaction.

7. (Co)polymers obtainable by the process according to claim 6.

8. Aqueous dispersions containing at least one (co)polymer according to claim 1.

9. A process for treating leather or leather precursor which are obtained in the leather production process comprising contacting it with the polymers according to claim 1 or of dispersions of said (co)copolymer.

10. Leather containing a (co)polymer according to claim 1.

11. (Co)polymers according to claim 1, characterized in that R³ represents a saturated C₁-C₆₀-alkyl radical, in particular C₈-C₃₀-alkyl radical.

12. (Co)polymers according to claim 1, characterized in that R³ represents a saturated C₈-C₃₀-alkyl radical.

13. (Co)polymers according to claim 3, characterized in that the copolymer comprises more than 98% by weight, of structural units of the formula I and the polyether units.

14. (Co)polymers according to claim 3, characterized in that from 50 to 90 mol %, based on the sum of these two units, is being accounted for by structural units of the formula I.

15. (Co)polymers according to claim 3, characterized in that from 50 to 90 mol %, based on the sum of these two units, is being accounted for by structural units of the formula I.

16. (Co)polymers according to claim 3, characterized in that the sum of these two units, is being accounted for by structural units of the formula I of which at least 10 mol %, based on the units of the formula I correspond to those of the formula Ia.

17. (Co)polymers according to claim 3, characterized in that the sum of these two units, is being accounted for by structural units of the formula I of which at least 10 to 35 mol %, based on the units of the formula I correspond to those of the formula Ia.

18. (Co)polymers according to claim 3, characterized in that the sum of these two units; is being accounted for by structural units of the formula I of which at least 10 mol %, based on the units of the formula I correspond to those of formula Ib 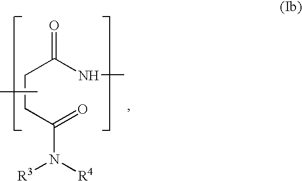
in which
R³ represents a hydrocarbon radical having C₁-C₆₀-atoms, and
R⁴ represents hydrogen or has the same meaning as R³.

19. (Co)polymers according to claim 3, characterized in that the sum of these two units, being accounted for by structural units of the formula I of which at least 10 to 35 mol %, based on the units of the formula I correspond, to those of formula Ib 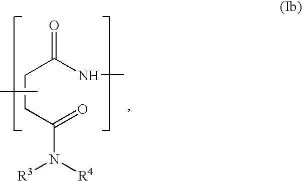
in which
R³ represents a hydrocarbon radical having C₁-C₆₀-atoms, and
R⁴ represents hydrogen or has the same meaning as R³.

20. (Co)polymers according to claim 4, characterized in that the copolymer comprises more than 98% by weight, of structural units of the formula I and the polyether units.

21. (Co)polymers according to claim 4, characterized in that
at least 10 to 50 mol %, the structural units of the formula I correspond to the formula Ia

22. (Co)polymers according to claim 4, characterized in that the structural units of formula I correspond to those of formula Ib 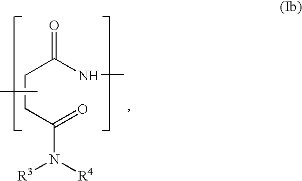
R³ represents a hydrocarbon radical having C₁-C₆₀-atoms, and
R⁴ represents hydrogen or has the same meaning as R³.

23. (Co)polymers according to claim 4, characterized in that structural units of formula I correspond to those of formula Id. 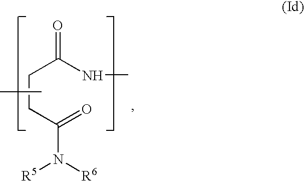
in which
R⁵ represents a radical which contains more than two ether groups derived from C₂-C₆-alkylene oxide units and which is optionally interrupted by urethane, carbonate, urea, biuret, allophanate, isocyanate, alkylene, cycloalkylene or aralkylene groups and
R⁶ represents hydrogen or has the meaning of R⁵.
